(19) United States
(12) Patent Application Publication
(10) Pub. No.: US 2024/0307121 A1
(43) Pub. Date: Sep. 19, 2024

(54) PREDICTIVE MODELING AND LESION ZONEIZATION AND INDEX/SCORE FROM PFA APPLICATION

(71) Applicant: Biosense Webster (Israel) Ltd., Yokneam (IL)

(72) Inventors: Paras PARIKH, Irvine, CA (US); Keshava DATTA, Chino Hills, CA (US); Tushar SHARMA, Irvine, CA (US)

(73) Assignee: Biosense Webster (Israel) Ltd., Yokneam (IL)

(21) Appl. No.: 18/409,554

(22) Filed: Jan. 10, 2024

Related U.S. Application Data
(60) Provisional application No. 63/440,275, filed on Jan. 20, 2023.

(51) Int. Cl. A61B 34/10; A61B 5/339; A61B 5/349; A61B 18/00; A61B 18/14; A61B 90/00; A61B 90/92; G16H 40/63

(52) U.S. Cl. CPC A61B 34/10; A61B 5/339; A61B 5/349; A61B 18/1492; A61B 90/92; G16H 40/63; A61B 2018/00351; A61B 2018/00577; A61B 2018/00613; A61B 2018/00904; A61B 2034/104; A61B 2090/061; A61B 2090/065

(57) ABSTRACT

Disclosed is a system and method to provide information about an ablation lesion zone (width or depth). Such information on the ablation lesion zone can be projected onto an electro-anatomical map based on the magnitude of the contact force, number of times energy were delivered to tissues and pre-clinical data.

### CROSS REFERENCE TO RELATED APPLICATIONS

This application claims the benefit of U.S. Provisional Application No. 63/440,275 entitled Predictive Modeling And Lesion Parametrization And Index/Score From PFA Application filed Jan. 20, 2023, which is incorporated by reference as if fully set forth.

### TECHNICAL FIELD

The present invention is directed to predictive modeling and lesion index/score from PFA application.

### BACKGROUND

Pulsed field ablation (PFA) is a newly applied technology to the field of cardiac electrophysiology. Lesions created using PFA technology are still relatively not well understood compared to RFA lesions. Currently physicians have no way of determining how deep the lesions will be from PFA lesions except for a nominal value based on pre-clinical work. The ability to help predict and understand the depth of lesion through the use of PFA will give physicians a more determinant endpoint and better guidance in terms of lesion creation and strategy.

### SUMMARY

A multi-parameter and/or single-parameter regression of pulsed field ablation (PFA) application parameter(s), and PFA lesion depth and width is used to determine zones of irreversible electroporation and reversible an ablation lesion zone (width or depth) projected onto the 3D electro-anatomical map. The regression correlates PFA application parameter(s) with lesion depth, width and spatial location based on distance from the PFA catheter and provides physicians information about the PFA lesions. Other parameters can include tissue wall thickness a type of tissues nearby (myocardium, smooth muscle etc.) as well as orientation of myocardial fibers.

After PFA application, a PFA “realistic” lesion can be projected onto the electro-anatomical map to provide visual of the PFA lesion including lesion width, lesion depth and cardiac tissue in the zone of irreversible electroporation and reversible electroporation. The PFA “realistic” lesion can be color-coded.

The lesions projected onto the cardiac anatomy (3D electro-anatomical map) from PFA application can be color coded and sized accordingly to the zone they are located in (irreversible (red color) and reversible (yellow color)) and based on parameters of PFA application. The size and spatial projection of the resulting lesion can be determined by the multiparametric regression created and validated form pre-clinical work that relates PFA parameters to lesions size and width.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1 illustrates a diagram of an exemplary system in which one or more features of the disclosure subject matter can be implemented according to one or more embodiments;

FIG. 2 illustrates a block diagram of an example system for catheter structure examination and optimization using medical procedure information according to one or more embodiments;

FIG. 3 illustrates a lasso-type ablation catheter that may be used in an exemplary system in which one or more features of the disclosure subject matter can be implemented according to one or more embodiments;

FIG. 4 illustrates a basket-type ablation catheter that may be used in an exemplary system in which one or more features of the disclosure subject matter can be implemented according to one or more embodiments;

FIG. 5 illustrates a large electrode that may be implemented with the basket-type ablation catheter of FIG. 4;

FIG. 6 illustrates a barrel electrode that may be implemented with the lasso-type ablation catheter of FIG. 3;

FIG. 7 illustrates the exponential plateau curve for other catheter designs;

FIG. 8 illustrates a visualization of the lesion created in the exemplary system in which one or more features of the disclosure subject matter can be implemented according to one or more embodiments;

FIG. 9 illustrates a sample tissue including a lesion of measured depth;

FIG. 10 illustrates a plot of the lesion depth as a function of electric field;

FIG. 11 illustrates a graphical depiction of the electrical fields associated with measuring lesion depth;

FIG. 12 illustrates a parametric curve based on the variable changed;

FIG. 13 illustrates a method according to an aspect of the present invention;

FIG. 14 illustrates the CF range per PFA dose;

FIG. 15 illustrates the impact of CF and PFA on lesion depth and lesion width;

FIG. 16 illustrates the impact of CF>5 g and PFA dose on lesion size;

FIG. 17 illustrates the relationship between CF and PFAI. PFAI and CF are linearly related for each attained PFAI value;

FIG. 18 illustrates the correlation between lesion depth and PFAI;

FIG. 19 illustrates the correlation between PFAI and the ventricular lesion depth;

FIG. 20 illustrates a chart of the depth regression;

FIG. 21 illustrates a chart of the depth interpolation regression;

FIG. 22 illustrates a chart of the width regression; and

FIG. 23 illustrates a chart of the width interpolation regression.

FIG. 24A illustrates an anatomical map of portions of a heart prior to ablation;

FIG. 24B illustrates an anatomical map with projection of the lesion and information relating the zone of lesion formed by the ablation; and

FIG. 25 illustrates a method according to an aspect of the present invention.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values such as +10% of the recited value, as an example. In addition, as used herein, the terms “patient,” “host,” “user,” and “subject” refer to any human or animal subject and are not intended to limit the systems or methods to human use, although use of the subject invention in a human patient represents a preferred embodiment.

A multi-parameter and/or single-parameter regression of pulsed field ablation (PFA) application parameter(s), and PFA lesion depth and width may be used to determine zones of irreversible electroporation and reversible an ablation lesion zone (width or depth) projected onto the 3D electro-anatomical map. The regression correlates PFA application parameter(s) with lesion depth, width and spatial location based on distance from the PFA catheter, and provides physicians information about the PFA lesions. Other parameters include tissue wall thickness, a type of tissues nearby (myocardium, smooth muscle etc.) and orientation of myocardial fibers.

After PFA application, a PFA “realistic” lesion can be projected onto the electro-anatomical map to provide visual of the PFA lesion including lesion width, lesion depth and cardiac tissue in the zone of irreversible electroporation and reversible electroporation. The PFA “realistic” lesion can be color-coded.

The lesions projected onto the cardiac anatomy (3D electro-anatomical map) from PFA application can be color coded and sized accordingly to the zone they are located in (irreversible (red color) and reversible (yellow color)) and based on parameters of PFA application. The size and spatial projection of the resulting lesion can be determined by the multiparametric regression created and validated form pre-clinical work that relates PFA parameters to lesions size and width.

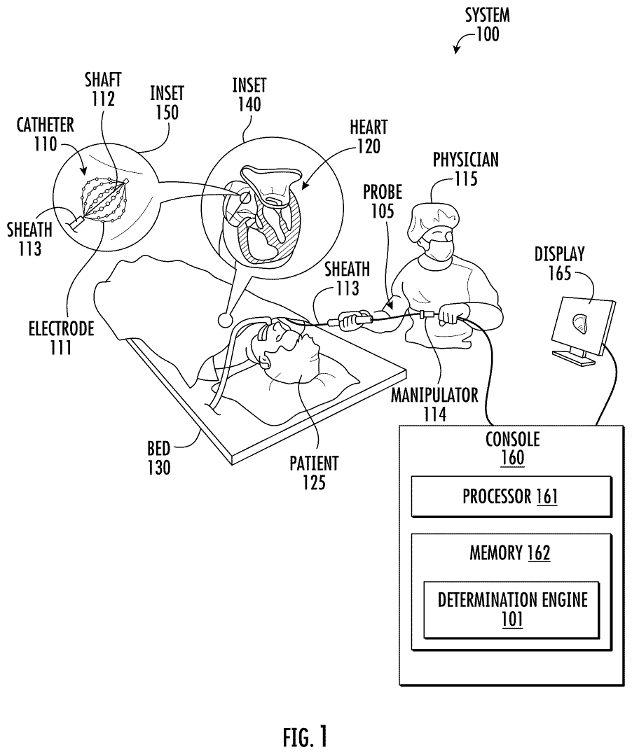

FIG. 1 is a diagram of a system 100 (e.g., a medical device equipment and/or an EP cardiac mapping system) in which one or more features of the subject matter herein can be implemented according to one or more embodiments. All or part of the system 100 can be used to collect information (e.g., biometric data and/or a training dataset) and/or used to implement a machine learning and/or an artificial intelligence algorithm (e.g., a determination engine 101) as described herein. The system 100, as illustrated, includes a probe 105 with a catheter 110 (including at least one electrode 111), a shaft 112, a sheath 113, and a manipulator 114. The system 100, as illustrated, is operated by a physician 115 (or a medical professional or clinician), operating on a heart 120 of a patient 125. Patient 125 may be located on a bed 130 (or a table). Insets 140 and 150 show the heart 120 and the catheter 110 in greater detail. The system 100 also, as illustrated, includes a console 160 (including one or more processor 161 and memory 162) and a display 165. Each element and/or item of the system 100 is representative of one or more of that element and/or that item. The example of the system 100 shown in FIG. 1 can be modified to implement the embodiments disclosed herein. The disclosed embodiments can similarly be applied using other system components and settings. Additionally, the system 100 can include additional components, such as elements for sensing electrical activity, wired or wireless connectors, processing and display devices, or the like.

The system 100 can be utilized to detect, diagnose, and/or treat cardiac conditions (e.g., using the determination engine 101). Cardiac conditions, such as cardiac arrhythmias, persist as common and dangerous medical ailments, especially in the aging population. For instance, the system 100 can be part of a surgical system (e.g., CARTO® system provided by Biosense Webster) that is configured to obtain biometric data (e.g., electro-anatomical and electrical measurements of a patient's organ, such as the heart 120) and perform a cardiac ablation procedure. More particularly, treatments for cardiac conditions such as cardiac arrhythmia often require obtaining a detailed mapping of cardiac tissue, chambers, veins, arteries and/or electrical pathways. For example, in performing a successful catheter ablation (as described herein) the cause of the cardiac arrhythmia is accurately located in a chamber of the heart 120. Such locating may be done via an EP investigation during which electrical potentials are detected spatially resolved with a mapping catheter (e.g., the catheter 110) introduced into the chamber of the heart 120. This EP investigation, the so-called electro-anatomical mapping, provides 3D mapping data which can be displayed on a monitor. In many cases, the mapping function and a treatment function (e.g., ablation) are provided by a single catheter or group of catheters such that the mapping catheter also operates as a treatment (e.g., ablation) catheter at the same time. In this case, the determination engine 101 outcomes or determinations can be directly stored and executed by the catheter 110.

In patients (e.g., the patient 125) with normal sinus rhythm (NSR), the heart (e.g., the heart 120), which includes atrial, ventricular, and excitatory conduction tissue, is electrically excited to beat in a synchronous, patterned fashion. The electrical excitement can be detected as intracardiac electrocardiogram (IC ECG) data or the like.

In patients (e.g., the patient 125) with a cardiac arrhythmia (e.g., atrial fibrillation or aFib), abnormal regions of cardiac tissue do not follow a synchronous beating cycle associated with normally conductive tissue, which is in contrast to patients with NSR. Instead, the abnormal regions of cardiac tissue aberrantly conduct to adjacent tissue, thereby disrupting the cardiac cycle into an asynchronous cardiac rhythm. The asynchronous cardiac rhythm can also be detected as the IC ECG data. Such abnormal conduction has been previously known to occur at various regions of the heart 120, for example, in the region of the sino-atrial (SA) node, along the conduction pathways of the atrioventricular (AV) node, or in the cardiac muscle tissue forming the walls of the ventricular and atrial cardiac chambers.

In support of the system 100 detecting, diagnosing, and/or treating cardiac conditions, the probe 105 can be navigated by the physician 115 into the heart 120 of the patient 125 lying on the bed 130. For instance, the physician 115 can insert the shaft 112 through the sheath 113, while manipulating a distal end of the shaft 112 using the manipulator 114 near the proximal end of the catheter 110 and/or deflection from the sheath 113. As shown in an inset 140, the catheter 110 can be fitted at the distal end of the shaft 112. The catheter 110 can be inserted through the sheath 113 in a collapsed state and can be then expanded within the heart 120.

Generally, electrical activity at a point in the heart 120 may be typically measured by advancing the catheter 110 containing an electrical sensor at or near its distal tip (e.g., the at least one electrode 111) to that point in the heart 120, contacting the tissue with the sensor and acquiring data at that point. One limitation with mapping a cardiac chamber using a catheter type containing only a single, distal tip electrode is the long period of time required to accumulate data on a point-by-point basis over the requisite number of points required for a detailed map of the chamber as a whole. Accordingly, multiple-electrode catheters (e.g., the catheter 110) have been developed to simultaneously measure electrical activity at multiple points in the heart chamber.

The catheter 110, which can include the at least one electrode 111 and a catheter needle coupled onto a body thereof, can be configured to obtain biometric data, such as electrical signals of an intra-body organ (e.g., the heart 120), and/or to ablate tissue areas of thereof (e.g., a cardiac chamber of the heart 120). The electrodes 111 are representative of any like elements, such as tracking coils, piezoelectric transducer, electrodes, or combination of elements configured to ablate the tissue areas or to obtain the biometric data. According to one or more embodiments, the catheter 110 can include one or more position sensors that used are to determine trajectory information. The trajectory information can be used to infer motion characteristics, such as the contractility of the tissue.

Biometric data (e.g., patient biometrics, patient data, or patient biometric data) can include one or more of local time activations (LATs), electrical activity, topology, bipolar mapping, reference activity, ventricle activity, dominant frequency, impedance, or the like. The LAT can be a point in time of a threshold activity corresponding to a local activation, calculated based on a normalized initial starting point. Electrical activity can be any applicable electrical signals that can be measured based on one or more thresholds and can be sensed and/or augmented based on signal to noise ratios and/or other filters. A topology can correspond to the physical structure of a body part or a portion of a body part and can correspond to changes in the physical structure relative to different parts of the body part or relative to different body parts. A dominant frequency can be a frequency or a range of frequencies that is prevalent at a portion of a body part and can be different in different portions of the same body part. For example, the dominant frequency of a PV of a heart can be different than the dominant frequency of the right atrium of the same heart. Impedance can be the resistance measurement at a given area of a body part.

Examples of biometric data include, but are not limited to, patient identification data, IC ECG data, bipolar intracardiac reference signals, electro-anatomical and electrical measurements, trajectory information, body surface (BS) ECG data, historical data, brain biometrics, blood pressure data, ultrasound signals, radio signals, audio signals, a two- or three-dimensional image data, blood glucose data, and temperature data. The biometrics data can be used, generally, to monitor, diagnosis, and treatment any number of various diseases, such as cardiovascular diseases (e.g., arrhythmias, cardiomyopathy, and coronary artery disease) and autoimmune diseases (e.g., type I and type II diabetes). Note that BS ECG data can include data and signals collected from electrodes on a surface of a patient, IC ECG data can include data and signals collected from electrodes within the patient, and ablation data can include data and signals collected from tissue that has been ablated. Further, BS ECG data, IC ECG data, and ablation data, along with catheter electrode position data, can be derived from one or more procedure recordings.

For example, the catheter 110 can use the electrodes 111 to implement intravascular ultrasound and/or MRI catheterization to image the heart 120 (e.g., obtain and process the biometric data). Inset 150 shows the catheter 110 in an enlarged view, inside a cardiac chamber of the heart 120. Although the catheter 110 is shown to be a point catheter, it will be understood that any shape that includes one or more electrodes 111 can be used to implement the embodiments disclosed herein.

Examples of the catheter 106 include, but are not limited to, a linear catheter with multiple electrodes, a balloon catheter including electrodes dispersed on multiple spines that shape the balloon, a lasso or loop catheter with multiple electrodes, or any other applicable shape (e.g., basket catheter, a multi-arm catheter, etc.). Linear catheters can be fully or partially elastic such that it can twist, bend, and or otherwise change its shape based on received signal and/or based on application of an external force (e.g., cardiac tissue) on the linear catheter. The balloon catheter can be designed such that when deployed into a patient's body, its electrodes can be held in intimate contact against an endocardial surface. As an example, a balloon catheter can be inserted into a lumen, such as a pulmonary vein (PV). The balloon catheter can be inserted into the PV in a deflated state, such that the balloon catheter does not occupy its maximum volume while being inserted into the PV. The balloon catheter can expand while inside the PV, such that those electrodes on the balloon catheter are in contact with an entire circular section of the PV. Such contact with an entire circular section of the PV, or any other lumen, can enable efficient imaging and/or ablation.

According to other examples, body patches and/or body surface electrodes may also be positioned on or proximate to a body of the patient 125. The catheter 110 with the one or more electrodes 111 can be positioned within the body (e.g., within the heart 120) and a position of the catheter 110 can be determined by the system 100 based on signals transmitted and received between the one or more electrodes 111 of the catheter 110 and the body patches and/or body surface electrodes. Additionally, the electrodes 111 can sense the biometric data (e.g., LAT values) from within the body of the patient 125 (e.g., within the heart 120). The biometric data can be associated with the determined position of the catheter 110 such that a rendering of the patient's body part (e.g., the heart 120) can be displayed and show the biometric data overlaid on a shape of the body part.

The probe 105 and other items of the system 100 can be connected to the console 160. The console 160 can include any computing device, which employs the machine learning and/or an artificial intelligence algorithm (represented as the determination engine 101). According to an embodiment, the console 160 includes the one or more processors 161 (any computing hardware) and the memory 162 (any non-transitory tangible media), where the one or more processors 161 execute computer instructions with respect the determination engine 101 and the memory 162 stores these instructions for execution by the one or more processors 161. For instance, the console 160 can be configured to receive and process the biometric data and determine if a given tissue area conducts electricity. In some embodiments, the console 160 can be further programmed by the determination engine 101 to carry out the functions of acquiring medical procedure information; examining the medical procedure information using a machine learning tool to discover data and catheter structure criteria; and optimizing, using the machine learning tool, a catheter structure for a procedure based on the data and the catheter structure criteria. According to one or more embodiments, the determination engine 101 can be external to the console 160 and can be located, for example, in the catheter 110, in an external device, in a mobile device, in a cloud-based device, or can be a standalone processor. In this regard, the determination engine 101 can be transferable/downloaded in electronic form, over a network.

In accordance with one or more embodiments, the determination engine 1010 can capture information from different procedures/sites, apply artificial intelligence to build an artificial intelligence model, and use the artificial intelligence model to build a recommendation map based on the different procedure/site parameters (e.g., at least part of an electro-anatomical structure, a specific procedure, and a physician experience) and to recommend a desired catheter, or a catheter with specific parameters and dimensions, to be used in a specific case. In accordance with one or more embodiments, the determination engine 101 can capture information from different procedures/sites, apply artificial intelligence to build an artificial intelligence model, and use the artificial intelligence model to analyze the different procedure/site parameters that contribute to a better procedure for specific catheter (e.g., and guide the physician 115 how to improve the catheter 110 usage). Also, the artificial intelligence model can be an iterative process that changes the catheter structure (virtually) to determine the optimal catheter structure.

In an example, the console 160 can be any computing device, as noted herein, including software (e.g., the determination engine 101) and/or hardware (e.g., the processor 161 and the memory 162), such as a general-purpose computer, with suitable front end and interface circuits for transmitting and receiving signals to and from the probe 105, as well as for controlling the other components of the system 100. For example, the front end and interface circuits include input/output (I/O) communication interfaces that enables the console 160 to receive signals from and/or transfer signals to the at least one electrode 111. The console 160 can include real-time noise reduction circuitry typically configured as a field programmable gate array (FPGA), followed by an analog-to-digital (A/D) ECG or electrocardiogramactromyogram (EMG) signal conversion integrated circuit. The console 160 can pass the signal from an A/D ECG or EMG circuit to another processor and/or can be programmed to perform one or more functions disclosed herein.

The display 165, which can be any electronic device for the visual presentation of the biometric data, is connected to the console 160. According to an embodiment, during a procedure, the console 160 can facilitate on the display 165 a presentation of a body part rendering to the physician 115 and store data representing the body part rendering in the memory 162. For instance, maps depicting motion characteristics can be rendered/constructed based on the trajectory information sampled at a sufficient number of points in the heart 120. As an example, the display 165 can include a touchscreen that can be configured to accept inputs from the medical professional 115, in addition to presenting the body part rendering.

In some embodiments, the physician 115 can manipulate the elements of the system 100 and/or the body part rendering using one or more input devices, such as a touch pad, a mouse, a keyboard, a gesture recognition apparatus, or the like. For example, an input device can be used to change a position of the catheter 110, such that rendering is updated. The display 165 can be located at a same location or a remote location, such as a separate hospital or in separate healthcare provider networks.

According to one or more embodiments, the system 100 can also obtain the biometric data using ultrasound, computed tomography (CT), MRI, or other medical imaging techniques utilizing the catheter 110 or other medical equipment. For instance, the system 100 can obtain ECG data and/or electro-anatomical and electrical measurements of the heart 120 (e.g., the biometric data) using one or more catheters 110 or other sensors. More particularly, the console 160 can be connected, by a cable, for example, to BS electrodes, which include adhesive skin patches affixed to the patient 125. The BS electrodes can procure/generate the biometric data in the form of the BS ECG data. For instance, the processor 161 can determine position coordinates of the catheter 110 inside the body part (e.g., the heart 120) of the patient 125. The position coordinates may be based on impedances or electromagnetic fields measured between the body surface electrodes and the electrode 111 of the catheter 110 or other electromagnetic components. Additionally, or alternatively, location pads may be located on a surface of the bed 130 and may be separate from the bed 130. The biometric data can be transmitted to the console 160 and stored in the memory 162. Additionally, or alternatively, the biometric data may be transmitted to a server, which may be local or remote, using a network as further described herein.

According to one or more embodiments, the catheter 110 may be configured to ablate tissue areas of a cardiac chamber of the heart 120. Inset 150 shows the catheter 110 in an enlarged view, inside a cardiac chamber of the heart 120. For instance, ablation electrodes, such as the at least one electrode 111, may be configured to provide energy to tissue areas of an intra-body organ (e.g., the heart 120). The energy may be thermal energy and may cause damage to the tissue area starting from the surface of the tissue area and extending into the thickness of the tissue area. The biometric data with respect to ablation procedures (e.g., ablation tissues, ablation locations, etc.) can be considered ablation data.

According to an example, with respect to obtaining the biometric data, a multi-electrode catheter (e.g., the catheter 110) can be advanced into a chamber of the heart 120. Anteroposterior (AP) and lateral fluorograms can be obtained to establish the position and orientation of each of the electrodes. ECGs can be recorded from each of the electrodes 111 in contact with a cardiac surface relative to a temporal reference, such as the onset of the P-wave in sinus rhythm from a BS ECG. The system, as further disclosed herein, may differentiate between those electrodes that register electrical activity and those that do not due to absence of close proximity to the endocardial wall. After initial ECGs are recorded, the catheter may be repositioned, and fluorograms and ECGs may be recorded again. An electrical map (e.g., via cardiac mapping) can then be constructed from iterations of the process above.

Cardiac mapping can be implemented using one or more techniques. Generally, mapping of cardiac areas such as cardiac regions, tissue, veins, arteries and/or electrical pathways of the heart 120 may result in identifying problem areas such as scar tissue, arrhythmia sources (e.g., electric rotors), healthy areas, and the like. Cardiac areas may be mapped such that a visual rendering of the mapped cardiac areas is provided using the display 165, as further disclosed herein. Additionally, cardiac mapping (which is an example of heart imaging) may include mapping based on one or more modalities such as, but not limited to local activation time (LAT), an electrical activity, a topology, a bipolar mapping, a dominant frequency, or an impedance. Data (e.g., biometric data) corresponding to multiple modalities may be captured using a catheter (e.g., the catheter 110) inserted into a patient's body and may be provided for rendering at the same time or at different times based on corresponding settings and/or preferences of the physician 115.

As an example of a first technique, cardiac mapping may be implemented by sensing an electrical property of heart tissue, for example, LAT, as a function of the precise location within the heart 120. The corresponding data (e.g., biometric data) may be acquired with one or more catheters (e.g., the catheter 110) that are advanced into the heart 1120 and that have electrical and location sensors (e.g., the electrodes 111) in their distal tips. As specific examples, location and electrical activity may be initially measured on about 10 to about 20 points on the interior surface of the heart 120. These data points may be generally sufficient to generate a preliminary reconstruction or map of the cardiac surface to a satisfactory quality. The preliminary map may be combined with data taken at additional points to generate a more comprehensive map of the heart's electrical activity. In clinical settings, it is not uncommon to accumulate data at 100 or more sites to generate a detailed, comprehensive map of heart chamber electrical activity. The generated detailed map may serve as the basis for deciding on a therapeutic course of action, for example, such as tissue ablation as described herein, to alter the propagation of the heart's electrical activity and to restore normal heart rhythm.

Further, cardiac mapping can be generated based on detection of intracardiac electrical potential fields (e.g., which is an example of IC ECG data and/or bipolar intracardiac reference signals). A non-contact technique to simultaneously acquire a large amount of cardiac electrical information may be implemented. For example, a catheter type having a distal end portion may be provided with a series of sensor electrodes distributed over its surface and connected to insulated electrical conductors for connection to signal sensing and processing means. The size and shape of the end portion may be such that the electrodes are spaced substantially away from the wall of the cardiac chamber. Intracardiac potential fields may be detected during a single cardiac beat. According to an example, the sensor electrodes may be distributed on a series of circumferences lying in planes spaced from each other. These planes may be perpendicular to the major axis of the end portion of the catheter. At least two additional electrodes may be provided adjacent at the ends of the major axis of the end portion. As a more specific example, the catheter may include four circumferences with eight electrodes spaced equiangularly on each circumference. Accordingly, in this specific implementation, the catheter may include at least 34 electrodes (32 circumferential and 2 end electrodes).

As example of electrical or cardiac mapping, an EP cardiac mapping system and technique based on a non-contact and non-expanded multi-electrode catheter (e.g., the catheter 110) can be implemented. ECGs may be obtained with one or more catheters 110 having multiple electrodes (e.g., such as between 42 to 122 electrodes). According to this implementation, knowledge of the relative geometry of the probe and the endocardium can be obtained by an independent imaging modality, such as transesophageal echocardiography. After the independent imaging, non-contact electrodes may be used to measure cardiac surface potentials and construct maps therefrom (e.g., in some cases using bipolar intracardiac reference signals). This technique can include the following steps (after the independent imaging step): (a) measuring electrical potentials with a plurality of electrodes disposed on a probe positioned in the heart 120; (b) determining the geometric relationship of the probe surface and the endocardial surface and/or other reference; (c) generating a matrix of coefficients representing the geometric relationship of the probe surface and the endocardial surface; and (d) determining endocardial potentials based on the electrode potentials and the matrix of coefficients.

As another example of electrical or cardiac mapping, a technique and apparatus for mapping the electrical potential distribution of a heart chamber can be implemented. An intra-cardiac multi-electrode mapping catheter assembly can be inserted into the heart 120. The mapping catheter (e.g., the catheter 110) assembly can include a multi-electrode array with one or more integral reference electrodes (e.g., one or the electrodes 111) or a companion reference catheter.

According to one or more embodiments, the electrodes may be deployed in the form of a substantially spherical array, which may be spatially referenced to a point on the endocardial surface by the reference electrode or by the reference catheter this is brought into contact with the endocardial surface. The electrode array catheter may carry a number of individual electrode sites (e.g., at least 24). Additionally, this example technique may be implemented with knowledge of the location of each of the electrode sites on the array, as well as knowledge of the cardiac geometry. These locations are determined by a technique of impedance plethysmography.

In view of electrical or cardiac mapping and according to another example, the catheter 110 can be a heart mapping catheter assembly that may include an electrode array defining a number of electrode sites. The heart mapping catheter assembly can also include a lumen to accept a reference catheter having a distal tip electrode assembly that may be used to probe the heart wall. The map heart mapping catheter assembly can include a braid of insulated wires (e.g., having 24 to 64 wires in the braid), and each of the wires may be used to form electrode sites. The heart mapping catheter assembly may be readily positionable in the heart 120 to be used to acquire electrical activity information from a first set of non-contact electrode sites and/or a second set of in-contact electrode sites.

Further, according to another example, the catheter 110 that can implement mapping EP activity within the heart can include a distal tip that is adapted for delivery of a stimulating pulse for pacing the heart or an ablative electrode for ablating tissue in contact with the tip. This catheter 110 can further include at least one pair of orthogonal electrodes to generate a difference signal indicative of the local cardiac electrical activity adjacent the orthogonal electrodes.

As noted herein, the system 100 can be utilized to detect, diagnose, and/or treat cardiac conditions. In example operation, a process for measuring EP data in a heart chamber may be implemented by the system 100. The process may include, in part, positioning a set of active and passive electrodes into the heart 120, supplying current to the active electrodes, thereby generating an electric field in the heart chamber, and measuring the electric field at the passive electrode sites. The passive electrodes are contained in an array positioned on an inflatable balloon of a balloon catheter. In embodiments, the array is said to have from 60 to 64 electrodes.

As another example operation, cardiac mapping may be implemented by the system 100 using one or more ultrasound transducers. The ultrasound transducers may be inserted into a patient's heart 120 and may collect a plurality of ultrasound slices (e.g., two dimensional or three-dimensional slices) at various locations and orientations within the heart 120. The location and orientation of a given ultrasound transducer may be known and the collected ultrasound slices may be stored such that they can be displayed at a later time. One or more ultrasound slices corresponding to the position of the probe 105 (e.g., a treatment catheter shown as catheter 110) at the later time may be displayed and the probe 105 may be overlaid onto the one or more ultrasound slices.

In view of the system 100, it is noted that cardiac arrhythmias, including atrial arrhythmias, may be of a multiwavelet reentrant type, characterized by multiple asynchronous loops of electrical impulses that are scattered about the atrial chamber and are often self-propagating (e.g., another example of the IC ECG data). Additionally, or alternatively, to the multiwavelet reentrant type, cardiac arrhythmias may also have a focal origin, such as when an isolated region of tissue in an atrium fires autonomously in a rapid, repetitive fashion (e.g., another example of the IC ECG data). Ventricular tachycardia (V-tach or VT) is a tachycardia, or fast heart rhythm that originates in one of the ventricles of the heart. This may be a life-threatening arrhythmia because it may lead to ventricular fibrillation and sudden death.

For example, aFib occurs when the normal electrical impulses (e.g., another example of the IC ECG data) generated by the sinoatrial node are overwhelmed by disorganized electrical impulses (e.g., signal interference) that originate in the atria veins and PVs causing irregular impulses to be conducted to the ventricles. An irregular heartbeat results and may last from minutes to weeks, or even years. aFib is often a chronic condition that leads to a small increase in the risk of death often due to strokes. The first line of treatment for aFib is medication that either slows the heart rate or reverts the heart rhythm back to normal. Additionally, persons with aFib are often given anticoagulants to protect them from the risk of stroke. The use of such anticoagulants comes with its own risk of internal bleeding. In some patients, medication is not sufficient and aFib is deemed to be drug-refractory, i.e., untreatable with standard pharmacological interventions. Synchronized electrical cardioversion may be used to convert aFib to a normal heart rhythm. Additionally, or alternatively, aFib patients are treated by catheter ablation.

A catheter ablation-based treatment may include mapping the electrical properties of heart tissue, especially the endocardium and the heart volume, and selectively ablating cardiac tissue by application of energy. Electrical or cardiac mapping (e.g., implemented by any EP cardiac mapping system and technique described herein) includes creating a map of electrical potentials (e.g., a voltage map) of the wave propagation along the heart tissue or a map of arrival times (e.g., a LAT map) to various tissue located points. Electrical or cardiac mapping (e.g., a cardiac map) may be used for detecting local heart tissue dysfunction. Ablations, such as those based on cardiac mapping, can cease or modify the propagation of unwanted electrical signals from one portion of the heart 120 to another.

The ablation process damages the unwanted electrical pathways by forming non-conducting lesions. Various energy delivery modalities have been disclosed for forming lesions, and include use of microwave, laser and more commonly, radiofrequency energies to create conduction blocks along the cardiac tissue wall. In a two-step procedure (e.g., mapping followed by ablation) electrical activity at points within the heart 120 is typically sensed and measured by advancing the catheter 110 containing one or more electrical sensors (e.g., electrodes 111) into the heart 120 and obtaining/acquiring data at a multiplicity of points (e.g., as biometric data generally, or as ECG data specifically). This ECG data is then utilized to select the endocardial target areas, at which ablation is to be performed.

Cardiac ablation and other cardiac EP procedures have become increasingly complex as clinicians treat challenging conditions such as atrial fibrillation and ventricular tachycardia. The treatment of complex arrhythmias can now rely on the use of three-dimensional (3D) mapping systems to reconstruct the anatomy of the heart chamber of interest. In this regard, the determination engine 101 employed by the system 100 manipulates and evaluates the biometric data generally, or the ECG data specifically, to produce improved tissue data that enables more accurate diagnosis, images, scans, and/or maps for treating an abnormal heartbeat or arrhythmia. For example, cardiologists rely upon software, such as the Complex Fractionated Atrial Electrograms (CFAE) module of the CARTOR 3 3D mapping system, produced by Biosense Webster, Inc. (Diamond Bar, Calif.), to generate and analyze ECG data. The determination engine 101 of the system 100 enhances this software to generate and analyze the improved biometric data, which further provide multiple pieces of information regarding EP properties of the heart 120 (including the scar tissue) that represent cardiac substrates (electro-anatomical and functional) of aFib.

Accordingly, the system 100 can implement a 3D mapping system, such as CARTOR 3 3D mapping system, to localize the potential arrhythmogenic substrate of the cardiomyopathy in terms of abnormal ECG detection. The substrate linked to these cardiac conditions is related to the presence of fragmented and prolonged ECGs in the endocardial and/or epicardial layers of the ventricular chambers (right and left). In general, abnormal tissue is characterized by low-voltage ECGs. However, initial clinical experience in endo-epicardial mapping indicates that areas of low-voltage are not always present as the sole arrhythmogenic mechanism in such patients. In fact, areas of low or medium voltage may exhibit ECG fragmentation and prolonged activities during sinus rhythm, which corresponds to the critical isthmus identified during sustained and organized ventricular arrhythmias, e.g., applies only to non-tolerated ventricular tachycardias. Moreover, in many cases, ECG fragmentation and prolonged activities are observed in the regions showing a normal or near-normal voltage amplitude (>1-1.5 mV). Although the latter areas may be evaluated according to the voltage amplitude, they cannot be considered as normal according to the intracardiac signal, thus representing a true arrhythmogenic substrate. The 3D mapping may be able to localize the arrhythmogenic substrate on the endocardial and/or epicardial layer of the right/left ventricle, which may vary in distribution according to the extension of the main disease.

As another example operation, cardiac mapping may be implemented by the system 100 using one or more multiple-electrode catheters (e.g., the catheter 110). Multiple-electrode catheters are used to stimulate and map electrical activity in the heart 120 and to ablate sites of aberrant electrical activity. In use, the multiple-electrode catheter is inserted into a major vein or artery, e.g., femoral artery, and then guided into the chamber of the heart 120. A typical ablation procedure involves the insertion of the catheter 110 having at least one electrode 111 at its distal end, into a heart chamber. A reference electrode is provided, taped to the skin of the patient or by means of a second catheter that is positioned in or near the heart or selected from one or the other electrodes 111 of the catheter 110. Radio frequency (RF) current is applied to a tip electrode 111 of the ablating catheter 110, and current flows through the media that surrounds it (e.g., blood and tissue) toward the reference electrode. The distribution of current depends on the amount of electrode surface in contact with the tissue as compared to blood, which has a higher conductivity than the tissue. Heating of the tissue occurs due to its electrical resistance. The tissue is heated sufficiently to cause cellular destruction in the cardiac tissue resulting in formation of a lesion within the cardiac tissue which is electrically non-conductive. During this process, heating of the tip electrode 111 also occurs as a result of conduction from the heated tissue to the electrode itself. If the electrode temperature becomes sufficiently high, possibly above 60 degrees Celsius, a thin transparent coating of dehydrated blood protein can form on the surface of the electrode 111. If the temperature continues to rise, this dehydrated layer can become progressively thicker resulting in blood coagulation on the electrode surface. Because dehydrated biological material has a higher electrical resistance than endocardial tissue, impedance to the flow of electrical energy into the tissue also increases. If the impedance increases sufficiently, an impedance rise occurs, and the catheter 110 must be removed from the body and the tip electrode 111 cleaned.

Information on the catheter may be recorded. This information may include, in addition to the elements described herein, tissue touch information, proximity touch information, the maneuverability of the catheter, the number of deflections of the catheter, for example.

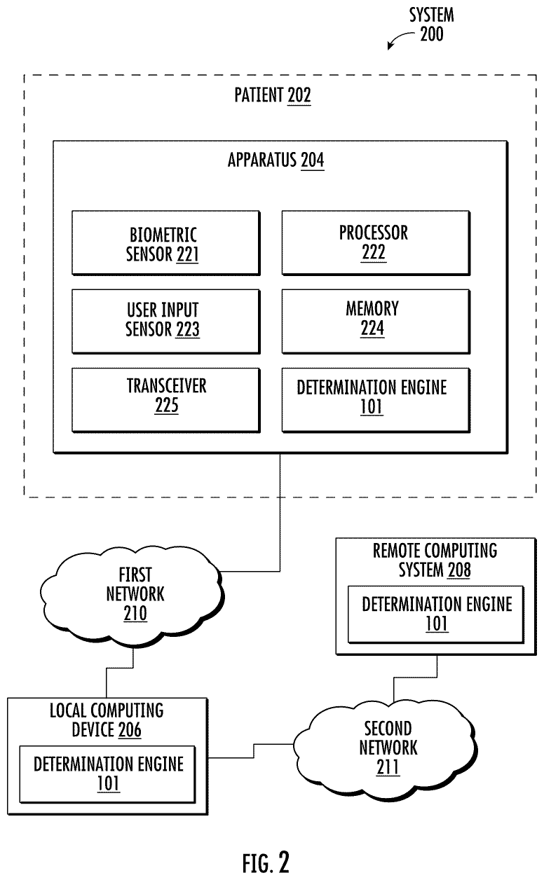

FIG. 2 illustrates a diagram of a system 200 in which one or more features of the disclosure subject matter can be implemented is illustrated according to one or more embodiments. The system 200 includes, in relation to a patient 202 (e.g., an example of the patient 125 of FIG. 1), an apparatus 204, a local computing device 206, a remote computing system 208, a first network 210, and a second network 211. Further, the apparatus 204 can include a biometric sensor 221 (e.g., an example of the catheter 110 of FIG. 1), a processor 222, a user input (UI) sensor 223, a memory 224, and a transceiver 225. The determination engine 101 of FIG. 1 is re-illustrated in FIG. 2 for ease of explanation and brevity.

According to an embodiment, the apparatus 204 can be an example of the system 100 of FIG. 1, where the apparatus 204 can include both components that are internal to the patient and components that are external to the patient. According to an embodiment, the apparatus 204 can be an apparatus that is external to the patient 202 that includes an attachable patch (e.g., that attaches to a patient's skin). According to another embodiment, the apparatus 204 can be internal to a body of the patient 202 (e.g., subcutaneously implantable), where the apparatus 204 can be inserted into the patient 202 via any applicable manner including orally injecting, surgical insertion via a vein or artery, an endoscopic procedure, or a laparoscopic procedure. According to an embodiment, while a single apparatus 204 is shown in FIG. 2, example systems may include a plurality of apparatuses.

Accordingly, the apparatus 204, the local computing device 206, and/or the remote computing system 208 can be programed to execute computer instructions with respect the determination engine 101. As an example, the memory 223 stores these instructions for execution by the processor 222 so that the apparatus 204 can receive and process the biometric data via the biometric sensor 201. In this way, the processor 22 and the memory 223 are representative of processors and memories of the local computing device 206 and/or the remote computing system 208.

The apparatus 204, local computing device 206, and/or the remote computing system 208 can be any combination of software and/or hardware that individually or collectively store, execute, and implement the determination engine 101 and functions thereof. Further, the apparatus 204, local computing device 206, and/or the remote computing system 208 can be an electronic, computer framework comprising and/or employing any number and combination of computing device and networks utilizing various communication technologies, as described herein. The apparatus 204, local computing device 206, and/or the remote computing system 208 can be easily scalable, extensible, and modular, with the ability to change to different services or reconfigure some features independently of others.

The networks 210 and 211 can be a wired network, a wireless network, or include one or more wired and wireless networks. According to an embodiment, the network 210 is an example of a short-range network (e.g., local area network (LAN), or personal area network (PAN)). Information can be sent, via the network 210, between the apparatus 204 and the local computing device 206 using any one of various short-range wireless communication protocols, such as Bluetooth, Wi-Fi, Zigbee, Z-Wave, near field communications (NFC), ultra-band, Zigbee, or infrared (IR). Further, the network 211 is an example of one or more of an Intranet, a local area network (LAN), a wide area network (WAN), a metropolitan area network (MAN), a direct connection or series of connections, a cellular telephone network, or any other network or medium capable of facilitating communication between the local computing device 206 and the remote computing system 208. Information can be sent, via the network 211, using any one of various long-range wireless communication protocols (e.g., TCP/IP, HTTP, 3G, 4G/LTE, or 5G/New Radio). For either network 210 and 211 wired connections can be implemented using Ethernet, Universal Serial Bus (USB), RJ-11 or any other wired connection and wireless connections can be implemented using Wi-Fi, WiMAX, and Bluetooth, infrared, cellular networks, satellite or any other wireless connection methodology.

In operation, the apparatus 204 can continually or periodically obtain, monitor, store, process, and communicate via network 210 the biometric data associated with the patient 202. Further, the apparatus 204, local computing device 206, and/the remote computing system 208 are in communication through the networks 210 and 211 (e.g., the local computing device 206 can be configured as a gateway between the apparatus 204 and the remote computing system 208). For instance, the apparatus 204 can be an example of the system 100 of FIG. 1 configured to communicate with the local computing device 206 via the network 210. The local computing device 206 can be, for example, a stationary/standalone device, a base station, a desktop/laptop computer, a smart phone, a smartwatch, a tablet, or other device configured to communicate with other devices via networks 211 and 210. The remote computing system 208, implemented as a physical server on or connected to the network 211 or as a virtual server in a public cloud computing provider (e.g., Amazon Web Services (AWS)®) of the network 211, can be configured to communicate with the local computing device 206 via the network 211. Thus, the biometric data associated with the patient 202 can be communicated throughout the system 200.

Elements of the apparatus 204 are now described. The biometric sensor 221 may include, for example, one or more transducers configured to convert one or more environmental conditions into an electrical signal, such that different types of biometric data are observed/obtained/acquired. For example, the biometric sensor 221 can include one or more of an electrode (e.g., the electrode 111 of FIG. 1), a temperature sensor (e.g., thermocouple), a blood pressure sensor, a blood glucose sensor, a blood oxygen sensor, a pH sensor, an accelerometer, and a microphone.

The processor 222, executing the determination engine 101, can be configured to receive, process, and manage the biometric data acquired by the biometric sensor 221, and communicate the biometric data to the memory 224 for storage and/or across the network 210 via the transceiver 225. Biometric data from one or more other apparatuses 204 can also be received by the processor 222 through the transceiver 225. Also, as described in more detail herein, the processor 222 may be configured to respond selectively to different tapping patterns (e.g., a single tap or a double tap) received from the UI sensor 223, such that different tasks of a patch (e.g., acquisition, storing, or transmission of data) can be activated based on the detected pattern. In some embodiments, the processor 222 can generate audible feedback with respect to detecting a gesture.

The UI sensor 223 includes, for example, a piezoelectric sensor or a capacitive sensor configured to receive a user input, such as a tapping or touching. For example, the UI sensor 223 can be controlled to implement a capacitive coupling, in response to tapping or touching a surface of the apparatus 204 by the patient 202. Gesture recognition may be implemented via any one of various capacitive types, such as resistive capacitive, surface capacitive, projected capacitive, surface acoustic wave, piezoelectric and infra-red touching. Capacitive sensors may be disposed at a small area or over a length of the surface, such that the tapping or touching of the surface activates the monitoring device.

The memory 224 is any non-transitory tangible media, such as magnetic, optical, or electronic memory (e.g., any suitable volatile and/or non-volatile memory, such as random-access memory or a hard disk drive). The memory 224 stores the computer instructions for execution by the processor 222.

The transceiver 225 may include a separate transmitter and a separate receiver. Alternatively, the transceiver 225 may include a transmitter and receiver integrated into a single device.

In operation, the apparatus 204, utilizing the determination engine 101, observes/obtains the biometric data of the patient 202 via the biometric sensor 221, stores the biometric data in the memory, and shares this biometric data across the system 200 via the transceiver 225. The determination engine 101 can utilize models, neural networks, machine learning, and/or artificial intelligence to measure effectiveness of any catheter structure (e.g., once built) and to provide quantitative feedback, statistical feedback, and/or like feedback of the effectiveness of a particular diagnostic catheter, which will improve the catheter structural designs.

The biometric data and/or the testing or measurement data may include, for example, the outcome of the procedure, the software used during the procedure plus any variables or options included or used within the software, such as physician and/or patient permissions, the serial number of the catheter used, the position of the catheter, the contact force of the catheter, the demographics of the patient including the BMI, and the anomaly being treated. The biometric data and/or the testing or measurement data may include where the procedure is performed including environmental data and also where in the body of the patient, such as, the atrium or ventricle, for example. The arrangement of the electrodes and which electrodes are used in the procedure may be included. The biometric data and/or the testing or measurement data may include follow-up information received post-procedure.

A multi-parameter and/or single-parameter regression of PFA application parameter(s) and PFA lesion depth and width may be used to determine zones of irreversible electroporation and reversible an ablation lesion zone (width or depth) projected onto the 3D electro-anatomical map. The regression correlates PFA application parameter(s) with lesion depth, width and spatial location based on distance from the PFA catheter and provides physicians information about the PFA lesions. Other parameters include tissue wall thickness a type of tissues nearby (myocardium, smooth muscle etc.) and orientation of myocardial fibers.

After a PFA application, a PFA “realistic” lesion may be projected onto the electro-anatomical map to provide a visual depiction of the PFA lesion including lesion width, lesion depth and cardiac tissue in the zone of irreversible electroporation and reversible electroporation. The visual depiction may be color-coded.

The lesions projected onto the cardiac anatomy (3D electro-anatomical map) from the PFA application may be color coded and sized according to their located zone in irreversible (i.e., red color) and reversible (i.e., yellow color) and based on parameters of PFA application. The size and spatial projection of the resulting lesion may be determined by the multiparametric regression created and validated from pre-clinical work that relates PFA parameters to lesions size and width.

A model can be created for each catheter design and the model may be particular to the E-field created and the particular geometry of the electrodes on the catheter. This provides detail of the general model with other PFA catheter technologies where the initial model is represented by the E-field (Voltage) and the other parameters are measured based on pre-clinical and in-vivo data. This data may include distance form endocardium including information gathered from other sources of data collection (i.e. Carto, ICE etc.). For other applications with PFA the parameter bounds may be different and the number of electrodes may be different but the general equation holds with the key parameters adjusted accordingly.

As presented in the paper Fundamental Study on the Effects of Irreversible Electroporation Pulses on Blood Vessels with Application to Medical Treatment by Elad Maor, lesion and modeling can be predicted accurately. As illustrated in this paper, there is a three-dimensional simulation illustrating the cross section of two needles and the electric field around the needles across the lower layer. As illustrated, the plot provides the electric field larger than 900 V/cm due to potential difference of 800 V. The voltage threshold reached based on the calculations in the model at the threshold of 900V/cm represent that area where cell death occurred.

Further, the paper illustrates a two-dimensional simulation of two round electrodes with a potential difference of 800 V. The plot illustrates electric field larger than 900 V/cm. The 2D area in blue represents the area of cell death in the experiment. The area closest to the electrodes in the field is a higher voltage/cm than 900V/cm and represents the area of irreversible electroporation.

Further illustrated is a two-dimensional ablation of adherent vascular smooth muscle cells (VSMC). The area of dead cells surrounds both needles and correlates with the experiment. The accuracy of the modeling is shown illustrating the modeling and the bench testing of electroporation. Both methodologies evidence the same area of cell death around the electrodes where the E-field is created to support predictive as tool for identifying cell due to electroporation.

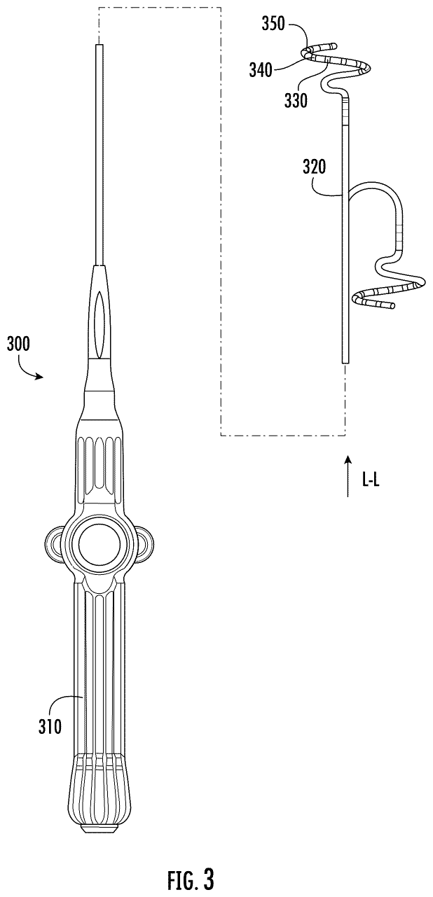

FIG. 3 illustrates a lasso-type ablation catheter 300 that may be used in an exemplary system in which one or more features of the disclosure subject matter can be implemented according to one or more embodiments. FIG. 3 shows an example of a loop catheter 300 (also referred to as a lasso catheter) including multiple electrodes 330, 340, 350 that may be used to map a cardiac area. Loop catheter 300 may be fully or partially elastic such that it can twist, bend, and or otherwise change its shape based on received signal and/or based on application of an external force (e.g., cardiac tissue) on the loop catheter 300. Loop catheter 300 may include a shaft 320 with the electrodes 330, 340, 350 affixed to the end of shaft 320. Shaft 320 may allow for the flexibility in whole or part. A handle 310 may be used to operate catheter 300. The electrodes may comprise, for example, ablation electrodes, electrical potential sensing electrodes, and/or any other suitable electrode type. The electrodes 330, 340, 350 may have a width between 1 mm and 4 mm, and are spaced between 1 mm and 10 mm apart. The electrodes 330, 340, 350 are connected to the connector at the proximal end of catheter 24 by wires (not shown) running through the catheter. Alternatively, other electrode configurations may be used. For example, the end section may include only ring electrodes, without a tip electrode.

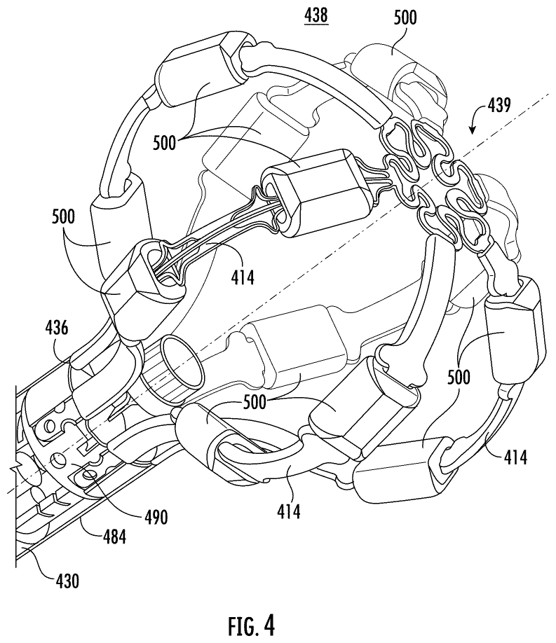

FIG. 4 illustrates a basket-type ablation catheter that may be used in an exemplary system in which one or more features of the disclosure subject matter can be implemented according to one or more embodiments. Experiments are conducted with a basket type ablation catheter substantially similar to the catheter end effector assembly 438 of FIG. 4. Basket assembly 438 includes a plurality of flexible spines 414 that are formed at the end of a tubular shaft 484 and are connected at both ends. During a medical procedure, medical professional 115 can deploy basket assembly 438 by extending tubular shaft 484 from insertion tube 430 (which is part of sheath 113) causing basket assembly 438 to exit insertion tube 430 and transition to the expanded form. Spines 414 may have elliptical e.g., circular, or rectangular that may appear to be flat cross-sections, and include a flexible, resilient material e.g., a shape-memory alloy such as nickel-titanium, also known as Nitinol forming a strut as will be described in greater detail herein. As shown in FIG. 4, basket assembly 438 has a proximal portion 436 with a distal end 439. The medical probe 105 can include a spine retention hub 490 that extends longitudinally from a distal end of tubular shaft 484 towards distal end 439 of basket assembly 438. As described supra, control console 160 includes irrigation module that delivers irrigation fluid to basket assembly 438 through tubular shaft 484.

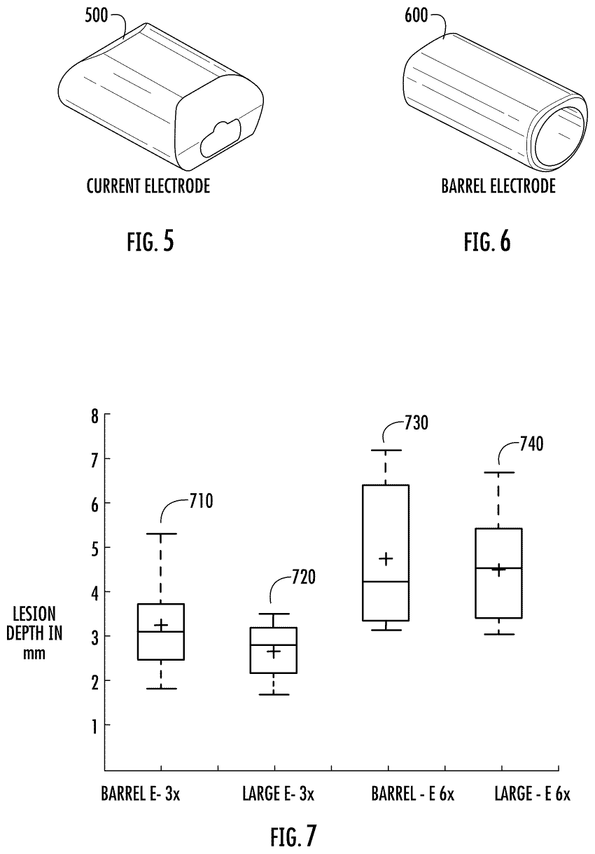

FIG. 5 illustrates a large electrode 500 that may be implemented with the basket-type ablation catheter of FIG. 4. FIG. 6 illustrates a barrel electrode that may be implemented with the lasso-type ablation catheter of FIG. 3. Two types of electrodes are used in the experiments: Large electrodes 500 such as one illustrated in FIG. 5 and smaller barrel type electrodes 600, shown in FIG. 6.

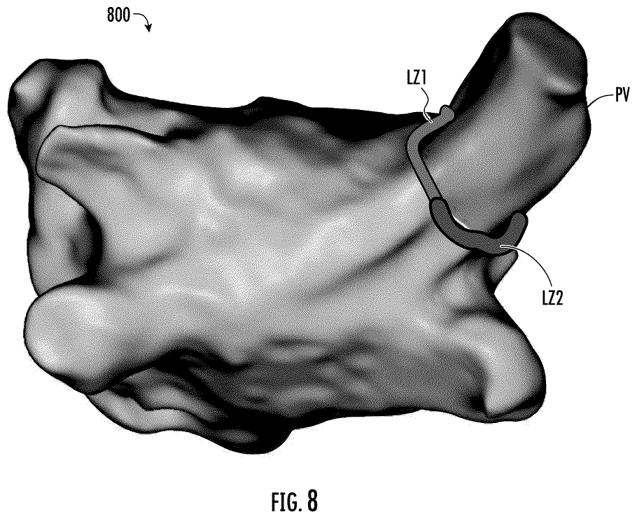

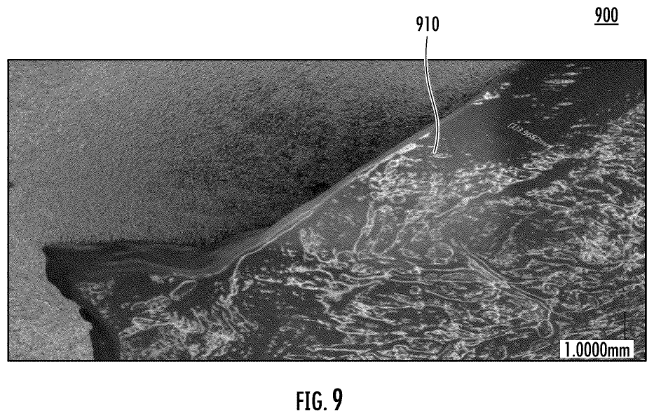

Ablations are conducted with these two types of electrodes (500, 600) with two different applications of ablation pulses: (1) 3 applications of predefined ablation pulses; and (2) 6 applications of the same predefined ablation pulses. The statistical data of these electrodes are shown in FIG. 7. A summary of the lesion depth and its deviation in the samples are summarized in Table 1.

TABLE 1
Ventricular Lesion Depth

| Type of IRE | Lesion depth for 3X | Lesion depth for 6X |
|---|---|---|
| Electrode | applications of pulses | applications of pulses |
| Large (500) | ~2.7 ± 0.7 mm | ~4.5 ± 1.4 mm |
| | (n = 13) (720) | (n = 9) (740) |
| Small (600) | ~2.9 ± 0.8 mm | ~4.7 ± 1.6 mm |
| | (n = 7) (710) | (n = 19) (730) |

From Table 1, (a) an increase in lesion depth correlates with increase in repetition number regardless of electrode size and (b) the smaller electrode seems to be slightly larger than large electrode in lesion depth but not a significant difference (p=0.67).

FIG. 8 illustrates a visualization 800 of the lesion created in the exemplary system in which one or more features of the disclosure subject matter can be implemented according to one or more embodiments. For example, a CARTO 3 system may be used to visualize the lesion to determine which lesion is better. As illustrated, lesion LZ1 s not as good as lesion LZ2. Lesion LZ2 is a better lesion. By example, lesion LZ2 is better because with feedback, the lesion with larger depth at the proper anatomical locations provides better durability for better efficacy long term.

FIG. 9 illustrates a depiction of a sample tissue 900 including a lesion 910 of measured depth. FIG. 9 provides an example of a lesion 910 creation using PFA in which there is a maximum depth and width of the PFA lesion. FIG. 9 illustrates an example of a lesion depth used for 3× application for PFA to inform and determine Equation 1 provided below.

Based in part on the experiments, a model of the curve of lesion size (LS) can be represented as in Equation 1:

$$LS = 6 * ( 1 - e - 0.538 {x}^{2} - 1.92 ) \qquad Equation 1$$

This formula for LS provides a specific formula related to a specific catheter and design. The electric field generated is specific to the catheter design and PFA formula. The curve provided by this formula may be shifted up or down based on the parameters described above. While this formula provides a specific example, it also illustrates the generic model employed for other catheters. As an example, other catheter designs may be provided with the same pulsed sequence. The increasingly asymptotically plateau curve for the other catheter designs is very similar. Therefore, the model may be based on an exponential plateau equation regardless of the number of variables used.

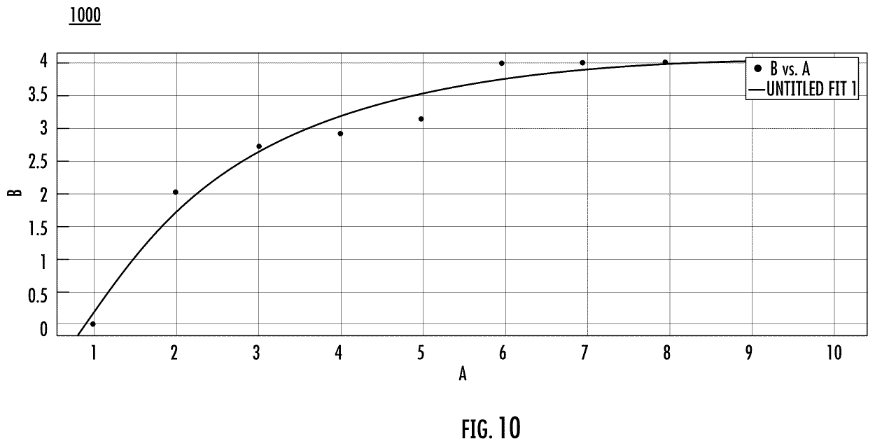

FIG. 10 illustrates a graphical representation 1000 of data collected from the Omnypulse catheter 400 of FIG. 4. The data of graph 1000 may be used to create a regression equation associating lesion depth (y-axis) with the number of PFA (x-axis).

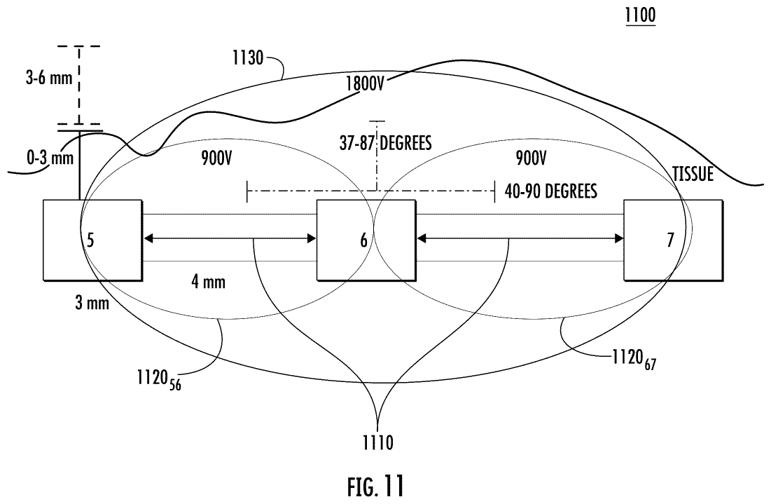

FIG. 11 illustrates a depiction 1100 of the electrical fields associated with measuring lesion depth. As illustrated there is a spacing 1110 between electrodes. In the illustration, this is provided as 4 mm. Neighboring electrodes provide an electric field 1120 associated between the pair (illustrated as 1120₅₆ between electrodes 5 and 6 and 1120₆₇ between electrodes 6 and 7, for example) as illustrated via the two smaller ovals. A larger electrical field 1130 is created surrounding the electrodes in general. This electrical field 1130 is depicted as the larger oval surrounding the electrodes. As is illustrated electrical field 1130 penetrates the tissue by approximately 1-3 mm allowing for measurement of the lesion associated with the tissue.

The measurement may occur using a Varipulse or Lasso system of FIG. 3 and the spacing 1110 between electrodes may be modified to be 1.5 mm. Further, Omnypulse of FIG. 4 or other catheter designs may be used to provide a spherical orb field 1130.

The parameters of the measurement including bounds may be PFA zone at risk: 0-10 mm (Particular to catheter shape and design and defined by E-field from COMSOL modeling), distance offset from endocardium (median value/zone): 0-10 mm (Construct with contact in mind and then have the statistical model and then the lesion depth is not achieved so predicter a lower coefficient or can be binary), Temperature/Conductivity: 35-90 degrees Celsius (Per Max, Max+)(Represents rate, depth and thermal effects), Dynamic Homogeneity of tissue: 0-1 (based on tissue type and # of applications), # of applications: 1-infinity, # of pulses/train:5, 7, 10 (min, Mod, max), max+(2200V), # of electrodes: 1-10 (zones 1-3,4-7,8-10) (capture in the model of distance), and Impedance range: (90-190 ohms): Take measurement of Bipolar impedance (160-190 ohms then the model works) while extrapolating other geometries of the electrodes to determine impedance. Contact of electrodes to tissue may be used as the number of electrodes in contact Range: 0 to Max # of electrodes. Contact force (CF) may be binary or quantitative with a range of 0-50 grams force. In addition, another variable that may be weighted in the equations is orientation of the catheter to the tissue or angle of orientation to tissue. This may include parallel, perpendicular, and oblique/diagonal. In addition, another parameter may include the number of applications or deliveries of the pulse and/or of the overall ablations performed in the singular location for the x, y, z coordinate on the tissue. Furthermore, FIG. 11 represents a visual schematic of the extent of the field and the specific parameters to determine lesion depth.

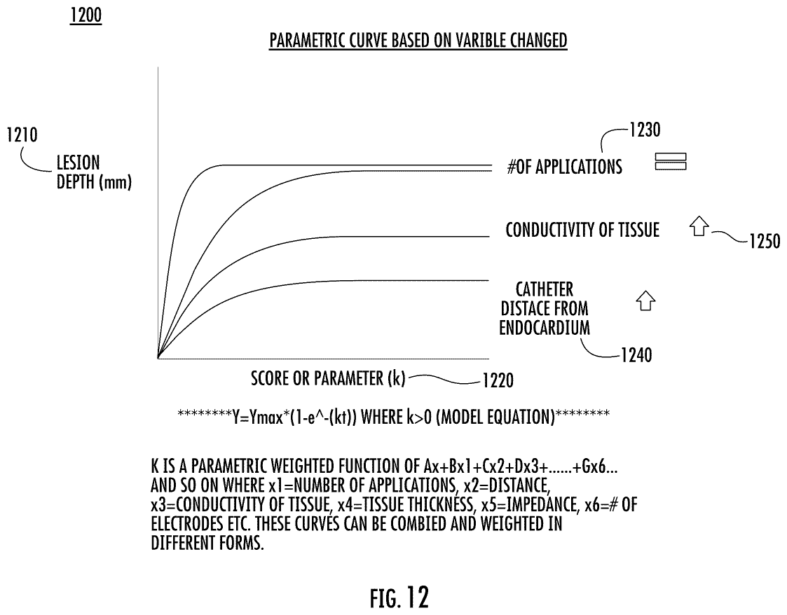

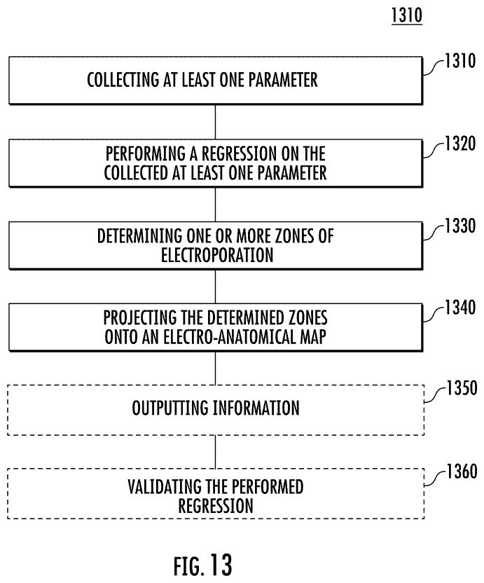

FIG. 12 illustrates a graph 1200 of a parametric curve based on the variable changed. This curve illustrates the lesion depth 1210 in mm as a function of score or parameter k 1220. k is a parametric function weighted function of Ax+Bx1+Cx2+Dx3+ . . . +Gx6 . . . . In an example, x1 equals the numbers of applications 1230, x2 equals the distance 1240, x3 equals the conductivity of tissue 1250, x4 equals the tissue thickness (not shown), x5 issues the impedance (not shown), x6 equals the number of electrodes (not shown), etc. The curves 1230-1250 and those discussed but not illustrated may be combined ad weighted in different forms.

Various parameters may be used. These include those discussed herein throughout. Parameters include number of applications which ranges from 1-12, contact force which ranges from 5-80 g after which there is no effect on the equations as they plateau, and distance form anatomy which ranges from 0-12 mm. Additionally, or alternatively, individually or in any combination, the following parameters may be used. These parameters may include, by way of non-limiting example only, voltage, current, impedance, conductivity, temperature, # of electrodes, resistance, # of pulses, pulse widths, time between applications, number of applications, tissue permeability, heterogeneity of tissue, electrical field density, frequency of ire, electrode geometry, and tissue mobility (cellular characteristic). These parameters may be included in the combination and weighting with curves 1230-1250 and those discussed but not illustrated may be combined ad weighted in different forms.

For example, temperature and distance to tissue may be correlated to thermal injury and directly correlated to zone of thermal injury. ECG attenuation overtime and different decays based on IRE vs. RE zones. Tissue contact, delivered current, number of applications may be correlated to lesion depth and provide value of initial E-field.

A singular and/or multiple parameter regression (any nonlinear, linear or parametric) that uses a combination of PFA parameters including but limited to voltage, current, number of pulses or pulse trains, number of PFA applications, time in between pulses, spatial E-field and distance of electrodes to tissue are related to the PFA lesion and that correlation can be used to highlight the zones of irreversible and reversible electroporation after application of the PFA lesion. The identification of the zones of IRE and RE on the cardiac tissue and relation of the above-mentioned parameters to lesion depth, width and location can be determined and validated from COMSOL simulations and pre-clinical studies to provide an assessment to physicians of the zones of cardiac tissue that have been affected by the PFA application. This parametric regression will be created such that the aforementioned parameters will be tested in a controlled pre-clinical model and validated where 1 or more of the parameters listed above (voltage, current, number of pulses, length of pulses, number of PFA applications, spatial E-field based on number of electrodes and distance of electrodes to tissue) will be characterized and correlated to the lesion created (lesions width, depth, and 3D spatial location). Any or all other parameters that are used/manipulated within the PFA ablation system can also be used to in the regression model to predict location and depth of lesion which can then be displayed on the 3D electro-anatomical map as irreversible or reversible electroporated tissue where irreversibly electroporated cardiac tissue is colored red and reversibly electroporated cardiac tissue is colored yellow on the 3D electro-anatomical map (CARTO).

Pre-clinical data aids to signify the irreversible and reversible ablation zones based on some type of serial imaging (i.e. MRI). In a model, the ability to identify irreversible a reversible ablation zones on tissue is provided. Further, the ability to use a mathematical model to predict lesions size which then predicts IRE and RE zones. The model may consider the specific catheter, the voltage, the number of electrodes and pulses the number of applications and location compared to endocardium as well as type of tissue and other cellular effects such as changing conductivity after each pulse. The model may include any nonlinear regression containing the parameters listed in the parameter's bounds can be used to supports the claims for creating a lesion score and predictive modeling of the PFA lesion. This model can be any nonlinear regression inclusive of all parameters listed in and parameter bounds and any model that uses any lesser parameters as well. Additional parameters could be added based on catheter type.

The use of an ECG response curve between zones of RE and IRE to determine the depth of lesion aids the model. The ECG may be utilized to correlate to preclinical work. There exists an association to different curves for IRE versus RE tissues when giving PFA to predict which ones result in IRE.

Modifications that can be done are to essentially change the type of parameters used (i.e. voltage, current etc.) and use all or any of the PFA parameters to create a correlation with PFA lesion, depth, width and spatial location of lesions on the cardiac tissue.

The model follows an exponential plateau regression where D=distance to myocardium, Max=Max lesion depth and k is the Dynamic cellular parameter. Equation 2 follows:

$$LS = ( Max - ( Max - Initial size ) * e ( - kx ) ) - Dist \qquad Equation 2$$

where x is the number of applications or number of pulses and Dist is the distance from the endocardium.

The model considers the voltage field based on the COMSOL modeling to generate LSmax. Distance is generated from CARTO or ICE or any form of data that could provide distance from the endocardium including Impedance. Initial size of the lesion is normally 0, although other values may also be used. k is a score or parameter that represents the cellular related changes to cause IRE as more pulses are given and the change in tissue structure, such as decreasing the time between pulses or applications may result in marginally increased lesion depth, and as well as potentially creating the lesion faster than pulses that have a larger time span. k is dependent on several parameter bounds including homogeneity of tissue, time between pulses or applications, the changing homogeneity of the electric field as more pulses are given, etc.

Intracardiac ECG may verify durability and depth between acute and chronic lesion measurements. Bench temperature data has also shown this kind of exponential plateau curve with temperatures plateauing as 60 degrees C. at 3 mm depth based on the particular PFA parameters (Number of applications, time delay between pulses and applications, Max voltage of the e-field and the current density).

The exponential plateau curve is realistic in modeling the data for the PFA lesions creation model. This has been illustrated with increased contact for PFA where the lesion growth curve achieved where the lesion depth plateaus at some point due to the limitations of the depth of the field.

The particularities of PFA lesion predication are illustrated to account for the differences in catheter design from company to company and the numerous parameters that exist in effect of creating the lesion not only at the catheter and generator level but also the cellular effects in-vivo during PFA ablation such as distance and surrounding tissue.

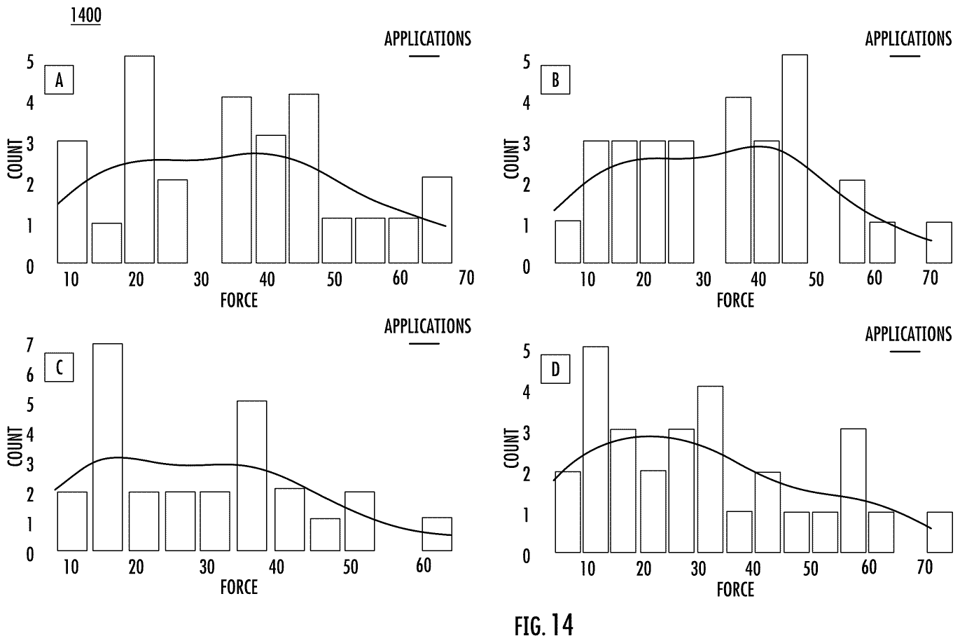

FIG. 13 illustrates a method 1300 according to an aspect of the present invention. The method 1300 for determining an ablation lesion zone (width or depth) projected onto an electro-anatomical map is provided.

The method 1300 includes collecting at least one ablation application parameter and at least ablation lesion depth, width and spatial location at 1310. The ablation may be a pulsed field ablation (PFA). The ablation application parameter may include tissue wall thickness, type of tissues nearby and orientation of myocardial fibers. The type of nearby tissues may include at least of myocardium and smooth muscle, for example.

The method 1300 includes performing a regression of the at least one ablation application parameter at 1320 to correlate the at least one ablation application parameter with ablation lesion depth, width and spatial location based on distance from the at least two electrodes to the biological tissues. The regression may include a multi-parameter regression. The regression may include a single-parameter regression. The performed regression may include pre-clinical work that relates the ablation parameters to the lesion depth and width.

The method 1300 includes determining one or more ablation zones at 1330. The electroporation may be irreversible. The electroporation may be reversible.

The method 1300 includes projecting the determined one or more ablation zones onto an electro-anatomical map at 1340. The electro-anatomical map may be three-dimensional. The projecting may provide a viewer of the map with a visual of the ablation lesion including lesion width, lesion depth and cardiac tissue in the zone of electroporation. The projecting may include color coding of the ablation lesion with the color-coding denoting at least one of irreversible electroporation and reversible electroporation. The size of the visual of the ablation lesion may correlate with the size of the lesion.

The method 1300 may also include outputting information about the ablation lesions at 1350. The method 1300 may also include validating at 1360 the performed regression pre-clinical work that relates the ablation parameters to at least one known lesion depth and width.

In conducting measurements for PFA ablations, a number of PFA applications are performed from x3, x6, x9, x12 and the CF ranged from 5-80 g. The CF is arranged in three strata including low (5-25 g), high (26-50 g), and very high (51-80 g). The definition of PFAI may be based on CF values and the number of PFA applications performed on each lesion. Equation 3 relates PFAI and lesion depth to the pulsed field ablation parameters of CF (contact force) and number of applications is provided below:

$$Expected Lesion Depth = PFAI / 100 \qquad Equation 3$$

Target ablations in the ventricles may be performed through pre-specified PFAI ranges (300, 450, and 600) and CF parameters (low or 5-25 g, high or 26-50 g, and finally, very high CF or 51-80 g). Each ventricle may be given 6 PFA ablations on average according to the ablation protocol as illustrated in Table 2 illustrating the study validation ablation parameters.

TABLE 2
Study validation ablation protocol
Study validation: Ablation Protocol

| | | | Count/per | | | | Count/per |
|---|---|---|---|---|---|---|---|
| | Index | Contact | chamber/ | | Index | Contact | chamber/ |
| | Target | Force | per animal | | Target | Force | per animal |
| RV | 300 | Low | 2 | LV | 450 | High | 2 |
| | 300 | High | 2 | | 600 | High | 2 |
| | 450 | Low | 2 | | 600 | Very High | 2 |

Lower PFAI values and CF thresholds may be used within the right ventricle to reduce transmural lesions which prevent accurate measurements of lesion depth at the higher PFAI values.

FIG. 14 illustrates four graphs 1400 of the CF range per PFA dose. The majority of CF values ranged from 10 to 50 g across all the PFA energy doses and are generally equally distributed between the x3 represented in FIG. 14A, x6 represented in FIG. 14B, x9 represented in FIG. 14C and x12 represented in FIG. 14D PFA dose. The number of PFA applications or PFA doses-x3, x6, x9, and x12 pulses—are equally distributed in the investigated study. The PFA applications are performed maintaining an average CF value of 32±17 g. As shown in FIG. 14 (collectively), similar CF values are maintained across the spectrum of different PFA doses. More specifically, CF values are kept within the low (5-25 g), high (26-50 g), and very high CF (51-80 g) range in 40%, 41%, and 15% of animals, respectively.

Lesion depth results are illustrated in Table 3 Study Characterization: correlation between PFA dose, Contact Force, and ventricular lesion depth.

TABLE 3
Study Characterization: correlation between PFA dose,
Contact Force, and ventricular lesion depth.

| | | Lesion Depth | | | |
|---|---|---|---|---|---|
| | | | Lesion Depth | | |
| APPLI- | Force | Sample | (Mean ± S.D) | | |
| CATIONS | range | size (n) | (mm) | min | max |
| 3 | 5-25 | 11 | 2.12 ± 0.72 | 0.901 | 3.05 |
| | 26-50 | 11 | 2.45 ± 0.51 | 1.71 | 3.043 |
| | 51-80 | 5 | 3.36 ± 1.43 | 2.25 | 5.6 |
| 6 | 5-25 | 12 | 2.66 ± 0.53 | 1.5 | 3.452 |
| | 26-50 | 13 | 3.84 ± 0.91 | 2.6 | 6.4 |
| | 51-80 | 4 | 3.69 ± 0.94 | 2.98 | 5.02 |
| 9 | 5-25 | 11 | 3.26 ± 0.77 | 1.981 | 4.512 |
| | 26-50 | 13 | 4.18 ± 1.01 | 2.8 | 5.633 |
| | 51-80 | 2 | 4.21 ± 1.00 | 3.508 | 4.92 |
| 12 | 5-25 | 11 | 3.93 ± 0.912 | 2.175 | 5.201 |
| | 26-50 | 12 | 4.67 ± 1.13 | 2.75 | 6.38 |
| | 51-80 | 6 | 5.33 ± 0.77 | 3.96 | 6.37 |

And the lesion width results are in Table 4 Study characterization: Correlation between PFA dose, Contact Force, and ventricular lesion width.

TABLE 4
Study characterization: Correlation between PFA dose,
Contact Force, and ventricular lesion width.

| | | Lesion Width | | | |
|---|---|---|---|---|---|
| | | | Lesion Depth | | |
| APPLI- | Force | Sample | (Mean ± S.D) | | |
| CATIONS | range | size (n) | (mm) | min | max |
| 3 | 5-25 | 10 | 8.2 ± 3.33 | 1.22 | 12.77 |
| | 26-50 | 11 | 9.55 ± 2.63 | 5.3 | 12.99 |
| | 51-80 | 5 | 11.9 ± 3.7 | 8.3 | 16 |
| 6 | 5-25 | 11 | 9.29 ± 3.67 | 4.6 | 14.86 |
| | 26-50 | 13 | 12.08 ± 2.58 | 8.27 | 17.33 |
| | 51-80 | 4 | 11.81 ± 3.29 | 9 | 16.51 |
| 9 | 5-25 | 9 | 14.07 ± 1.21 | 12.39 | 15.43 |
| | 26-50 | 12 | 14.51 ± 2.94 | 8.04 | 17.35 |
| | 51-80 | 2 | 13.48 ± 2.57 | 11.67 | 15.3 |
| 12 | 5-25 | 8 | 12.21 ± 1.75 | 8.42 | 14.38 |
| | 26-50 | 11 | 14.38 ± 2.6 | 10.69 | 19 |
| | 51-80 | 6 | 15.01 ± 2.81 | 10.49 | 19.26 |

The average lesion depth and width are 3.5±1.2 mm and 12.0±3.5 mm, respectively. The lesion depth spanned from 0.90 to 6.4 mm and ≥5 mm depth is achieved in 17% of the lesions out of which most (58%) performed with the highest PFA dose (x12). None of the performed lesions are transmural.

Lesion size is clearly influenced by both the number of PFA applications (i.e., PFA dose) and the CF values (catheter-tissue contact). In fact, considering the spectrum of PFA dose, the average lesion depth nearly doubled from 2.48±0.90 to 4.52±1.09 mm when x3 and x12 PFA dose are applied, respectively, i.e., p<0.005 from Table 3, with slightly wider lesions generally expected for higher PFA doses illustrated in Table 4. CF impacted lesion size regardless of the application numbers with the fixed number of applications, mean lesion depth increased over 1 mm from 10 g to 60 g of CF and consistently across application numbers. In this regard, applications performed within the low CF range (5-25 g) are associated with significantly shallower ventricular lesions (2.98±0.98 mm) compared to those carried out with high (26-50 g; 3.82±1.21 mm) and very high CF values (51-80 g; 4.23±1.30 mm) (p<0.05) in Table 3. Similar observations applied to lesion width in Table 4.

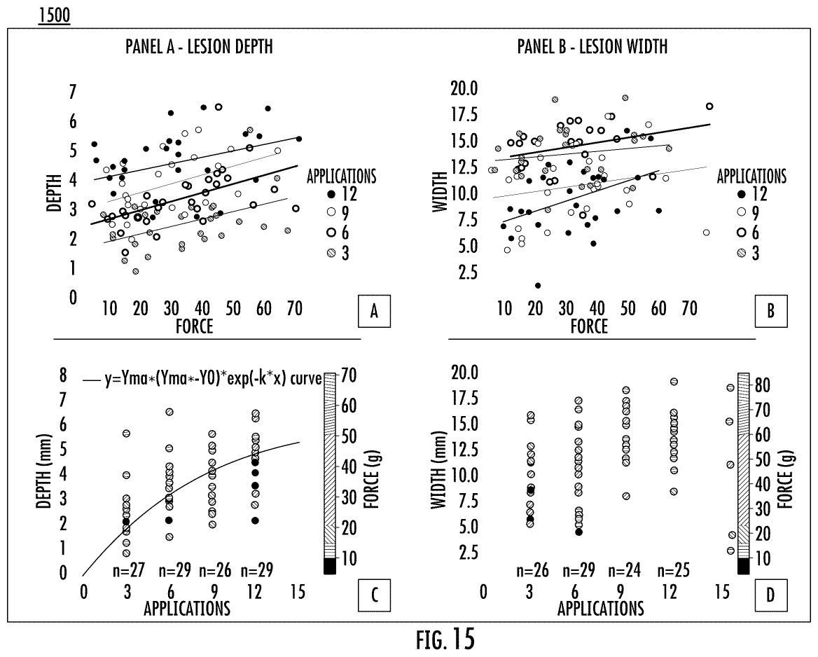

FIG. 15 illustrates several plots 1500 of the impact of CF and PFA on lesion depth Panel A and lesion width Panel B. For each PFA application, the lesion depth is illustrated in FIG. 15A and the lesion width illustrated in FIG. 15B are linearly related to CF values. An increase in the lesion size is realized when toggling from very low x3 to high dose PFA x12 provided that CF>5 g is warranted. The increase in lesion depth is illustrated in FIG. 15C and the increase in lesion width is illustrated in FIG. 15D. The interplay between CF and PFA to exponentially increase lesion depth is illustrated in FIG. 15C. The impact on lesion width illustrated in FIG. 15D is less clear.

Therefore, regression analyses may rule out any potential confounding effect of CF and PFA dose on lesion size as illustrated in FIG. 15A-D. While the relationship between CF and lesion depth (y=2.882+0.0209 x; r2=0.08; p=0.0024) in FIG. 15A and width (y=10.633+0.0422 x; r2=0.04; p=0.0043) FIG. 15B best fit on linear regression curves across the spectrum of PFA applications, the interplay between CF and PFA dose to impact on the ventricular lesion depth through a logarithmic relationship as illustrated in FIG. 15C. More than CF and PFA dose alone, it is the combination PFA energy with CF>5 g to determine remarkably deeper lesions. The role of CF and PFA dose on lesion width seemed less predictable as illustrated in FIG. 15D.

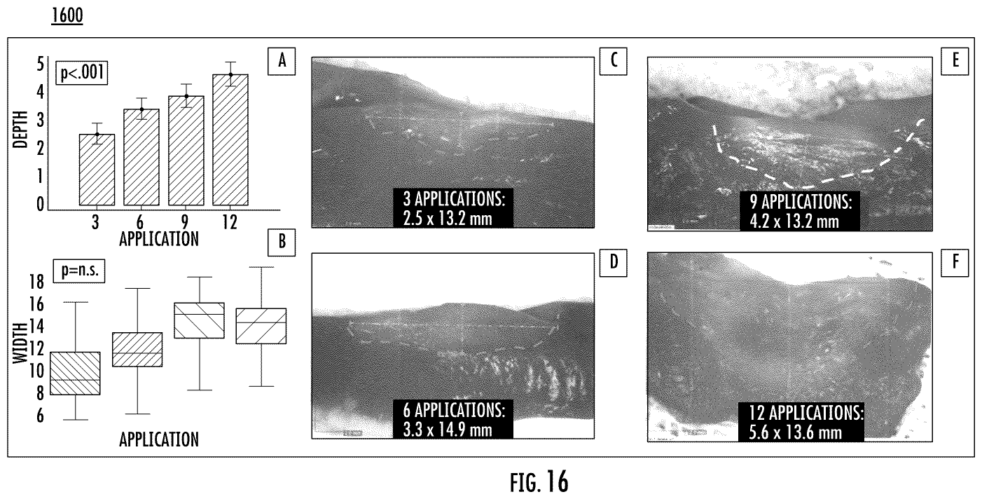

FIG. 16 illustrates several depictions 1600 of the impact of CF>5 g and PFA dose on lesion size. After achieving a good catheter contact (CF>5 g), progressively higher PFA doses are associated with deeper lesions as illustrated in FIG. 16A but with overlapping width as illustrated in FIG. 16B. The histologic sections are consistent with the data in FIGS. 16A and 16B as illustrated in FIG. 16C, 16D, 16E, 16F. FIG. 16C illustrates 3 application with a 2.5×13.2 mm lesion. FIG. 16D represents 6 application with a 3.3×14.9 mm lesion. FIG. 16E represents 9 applications with a 4.2 v 13.2 mm lesion. FIG. 16F represents 12 applications with 5.6×13.6 mm lesion. When the system is toggled from x3 to x12 PFA dose, a slight increase in lesion width occurs, and the average lesion depth doubled. Specifically, the average lesion width is 9.739889 mm with 3 applications, 11.19132 mm with 6 applications, 13.97311 mm with 9 applications, and 13.77438 mm with 12 application. The average lesion depth is 2.5 mm with 3 applications, 3.3 mm at 6 applications, 4.2 mm with 9 applications, and 5.6 mm with 12 applications. The lesion depth changed as evidenced in the data, but lesion width did not significantly change after 9 applications. The effects on lesion width and depth represent a weaker relationship between the lesion width and regression equations than the lesion depth because the dynamic range for the lesion width is smaller than the lesion depth.

Differently from lesion depth illustrated in FIG. 16A, progressively greater PFA applications applied with CF>5 g led to overlapping lesion width as illustrated in FIG. 16B. As it is demonstrated on histology sections illustrated in FIGS. 16C-F, depth nearly doubled when the system is toggled from low dose x3 PFA illustrated in FIG. 16C to high-dose PFAx12 pulses illustrated in FIG. 16F.

PFA lesions in the right and left ventricle, respectively are analyzed to illustrate if the PFAI (300, 450, and 600 range) may predict the actual lesion depth. Per ablation protocol of Table 2, 6.8±1.2 and 5.3±0.8 applications are assessed in the right and left ventricle, respectively, where 29 (34%) are within the 300 PFAI range (307±21, range: 268-343), 23 (31%) in the 450 (460±24, range: 424-554), and, finally, 21 (29%) in the 600 PFAI range (547±34, range: 475-590). As for CF values, 29 (40%), 30 (41%), and 14 (19%) of lesions are performed within the low (5-25 g), high (26-50 g), and very high (51-80 g) CF strata, respectively. In this data set, the average lesion depth is 3.6±1.0 mm (3.2±0.9 mm and 4.2±0.9 mm for the right and left ventricle, respectively) ranging from 1.7 to 6.6 mm and achieved with a mean PFAI of 424±105. Table 5 displays the average lesion depth associated with each attained PFAI range (i.e., 300, 450, and 600) and stratified according to the low, high, and very high CF datasets. Table 5 illustrates Study validation: Correlation between PFA dose, Contact Force, and ventricular lesion depth.

TABLE 5
Study validation: Correlation between PFA dose,
Contact Force, and ventricular lesion depth.

Lesion Depth

| | | | Lesion Depth | | |
|---|---|---|---|---|---|
| PFA | Force | Sample | (Mean ± S.D) | | |
| range | range | size (n) | (mm) | min | max |
| 300 | 5-25 | 15 | 2.80 ± 0.68 | 1.697 | 4.11 |
| | 26-50 | 12 | 2.92 ± 0.67 | 1.8 | 4.21 |
| | 51-80 | 2 | 2.89 ± 0.91 | 2.25 | 3.53 |
| 450 | 5-25 | 12 | 3.82 ± 0.79 | 2.825 | 5.188 |
| | 26-50 | 10 | 4.06 ± 0.71 | 3.1 | 5.561 |
| | 51-80 | 1 | 3.84 | 3.84 | 3.84 |
| 600 | 5-25 | 2 | 3.33 ± 1.87 | 2 | 4.65 |
| | 26-50 | 8 | 4.74 ± 0.97 | 3.394 | 6.582 |
| | 51-80 | 11 | 4.37 ± 0.64 | 3.421 | 5.462 |

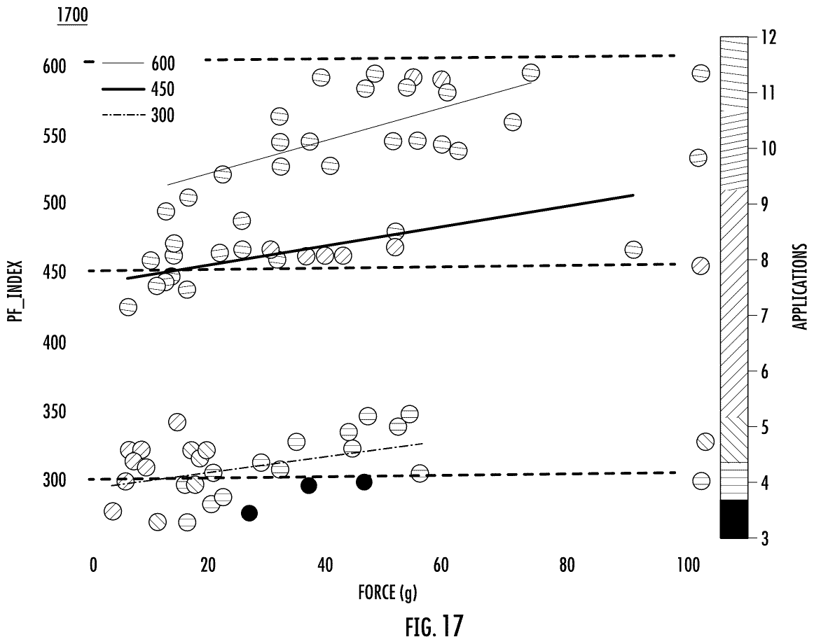

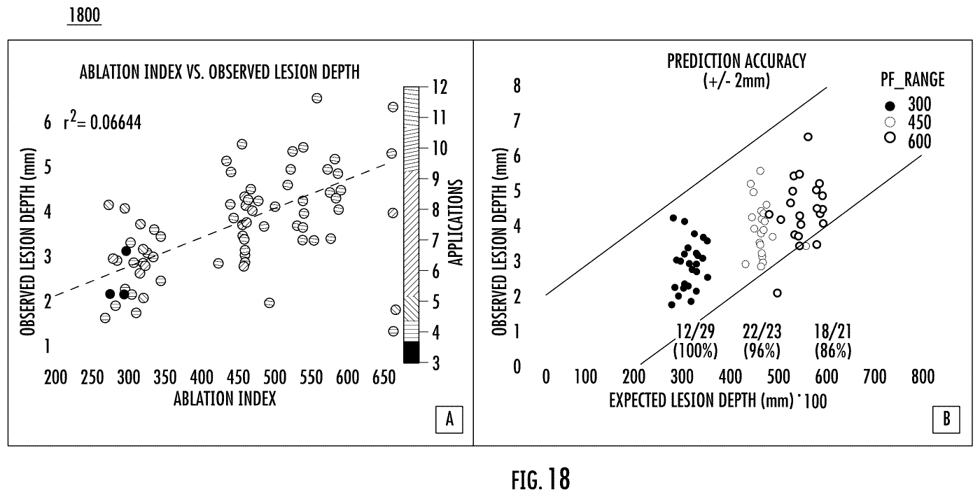

FIG. 17 illustrates graphically the relationship 1700 between CF and PFAI. PFAI and CF are linearly related for each attained PFAI value. PFAI and CF are linearly related with higher PFAI values (300, 450, and 600) leading to progressively deeper lesions (2.86±0.67, 3.92±0.73, and 4.41±0.94 mm, respectively; p<0.001).

FIG. 18 illustrates the correlation 1800 between lesion depth and PFAI. FIG. 18A illustrates the attained PFAI values plotted against the observed lesion depth histology. FIG. 18B illustrates the prediction accuracy of lesion depth compared to expected lesion depth. Lesion depth and PFAI are linearly related with more than 60% of the variation of the lesion depth defined by the variation of the PFAI parameter (r2-0.6644). The actual lesion depth may be plotted against the expected lesion depth (PFAI/1000). The actual lesion depth is predicted by the PFAI values attained during ablation with prediction accuracy of ±2 mm. The association between PFAI and lesion depth may be described using a linear regression model (y=0.923+0.640 x; r2=0.66; p<0.001) as illustrated in FIG. 18A where the actual ventricular lesion depth could be predicted for each lesion with an accuracy of ±2 mm by dividing the attained PFAI by a factor of 100 as illustrated in FIG. 18B.

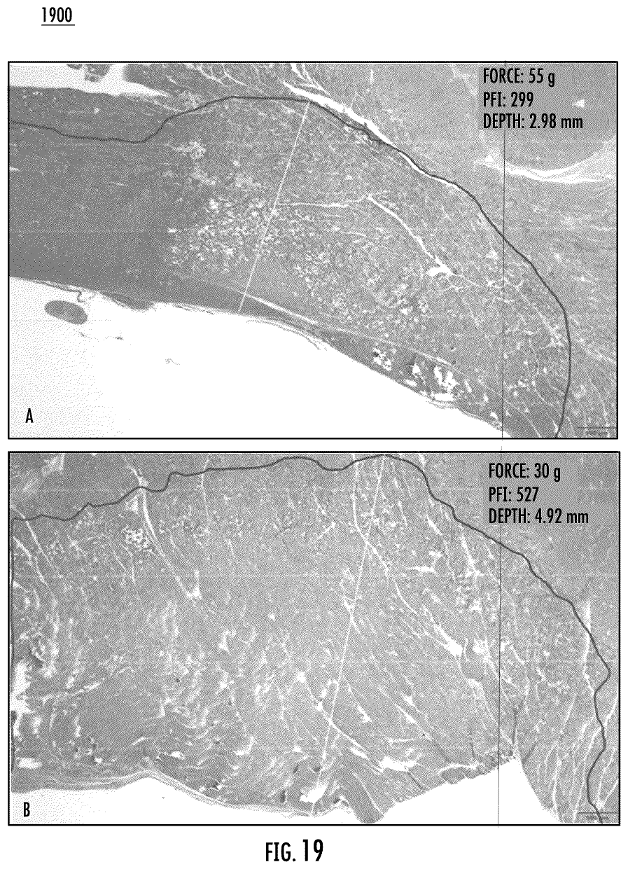

FIG. 19 illustrates two histologies 1900 providing the correlation between PFAI and the ventricular lesion depth. The PFAI is correlated with the average ventricular lesion depth through the formula described above (Lesion Depth=PFAI/100). Without regard to the contact force applied during the ablation. As illustrated in FIG. 19A a PFAI of 298 and FIG. 19B a PFAI of 527 provides insight on the prediction of the lesion depth through the PFAI value. Specifically, in FIG. 19A a force of 55 g, PFI of 299 provided a measured depth of 2.98 mm and FIG. 19B a force of 30 g, PFI of 527 provide a measured depth of 4.92 mm. The deeper lesion may be achieved by toggling from low dose to high dose PFA regardless of CF values.

The impact of CF and PFA dose on the ventricular lesion size undergoing PFA using the CF-sensing OMNYPULSE catheter is provided. Although both variables proved to be entailed in adequate lesion formation, more than CF and the number of PFA applications alone, it is their interplay to synergistically act on lesion formation. In fact, once adequate catheter-tissue contact is achieved (CF>5 g), toggling from low-dose to high-dose PFA led to remarkably deeper lesions with no clear effect on lesion width. The validation of these parameter through a brand-new formula utilized to guide PFA ablation—the PFAI—further helped to understand the impact of CF and the number of PFA applications on lesion size by predicting the actual ventricular lesion depth.

These results are even more important in the light of the lack of data on the optimal ablation parameters needed to achieve adequate lesion formation during VT ablation. In fact, unlike the field of AF ablation where the Ablation Index (AI) algorithm has been extensively investigated, there is very limited experience on the implementation of AI during VT ablation, and an equivalent index of lesion quality has not been hitherto investigated for PFA in this setting.

A new index—the PFAI—is provided to guide PFA in the ventricular chambers and to accurately predict the actual ventricular lesion depth.

The catheter-tissue contact is paramount in achieving adequately deep lesions, the PFA applications are performed with CF>5 g. Although recent evidence showed that none or minimal detectable lesions would result from PFA without good catheter-tissue contact and that an increase of 1-2 mm in distance of the catheter tip from the myocardial tissue would lead to a doubling of the energy necessary to create a 3-mm deep lesion.

As assessed on histology, the combined effect of PFA dose and CF during PFA provide a synergistic impact on the ventricular lesion size. The PFAI—a new parameter of lesion quality implementing PFA dose and CF—is enabled to predict the actual lesion size. For a given width of the lesion, the next point in the ablation may be calculated based on the actual data, and the location of the next point may be defined by the system.

Using a depth and width regression as illustrated in Equation 4:

$$V = Vm - ( Vm - V 0 ) * exp ( - ( b 0 * log ( n ) ) * x ) \qquad Equation 4$$

where V is the dependent variable (depth or width), x is the independent variable (force), n is the application number, Vm is the max value of V (plateau value), V0 is the initial value of V, and b0 is the parameter.

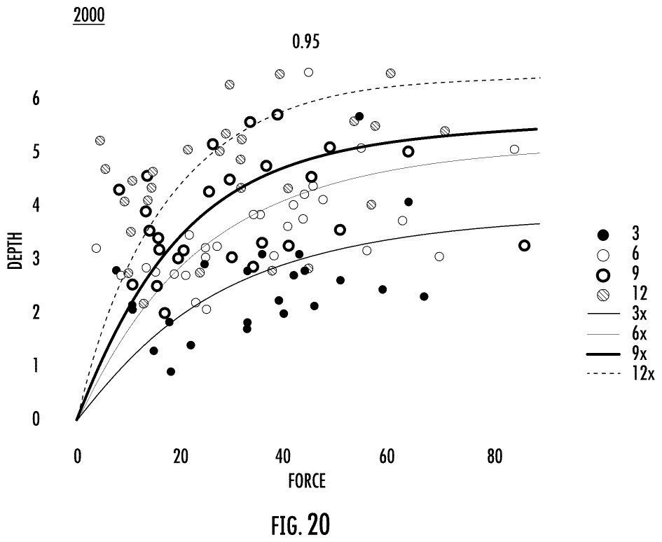

FIG. 20 illustrates a chart 2000 of the depth regression. In association with Table 6, below, providing the application numbers and b0 of the regression.

TABLE 6
Depth Regression

| Application | |
|---|---|
| of Energy | b0 |
| 3 | 0.033638628939413794 |
| 6 | 0.02288434395310522 |
| 9 | 0.02278212911071813 |
| 12 | 0.023497926794668167 |

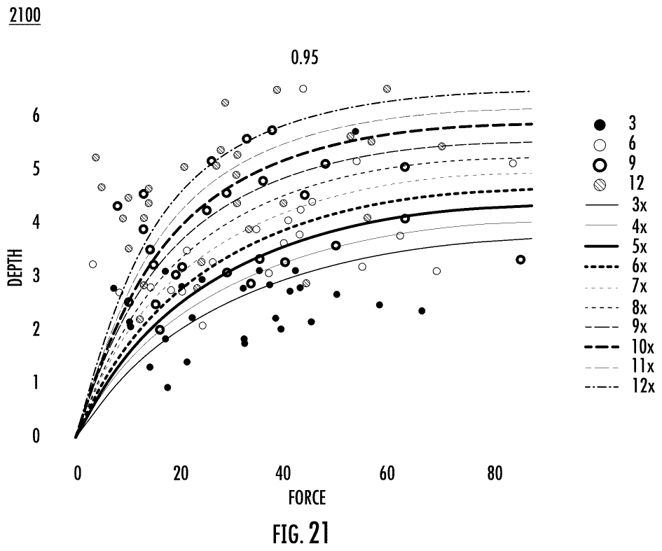

FIG. 21 illustrates a chart 2100 of the depth interpolation regression. In association with Table 7, below, providing the application numbers and b0 of the regression.

TABLE 7
Depth Interpolation Regression

| Application | |
|---|---|
| of Energy | b0 |
| 3 | 0.033638628939413794 |
| 4 | 0.028374221485542896 |
| 5 | 0.025918511701563538 |
| 6 | 0.024609021603300522 |
| 7 | 0.023882222737006107 |
| 8 | 0.02349278435895052 |
| 9 | 0.02331627106939869 |
| 10 | 0.02328265571093192 |
| 11 | 0.02334943845139342 |
| 12 | 0.023497926794668167 |

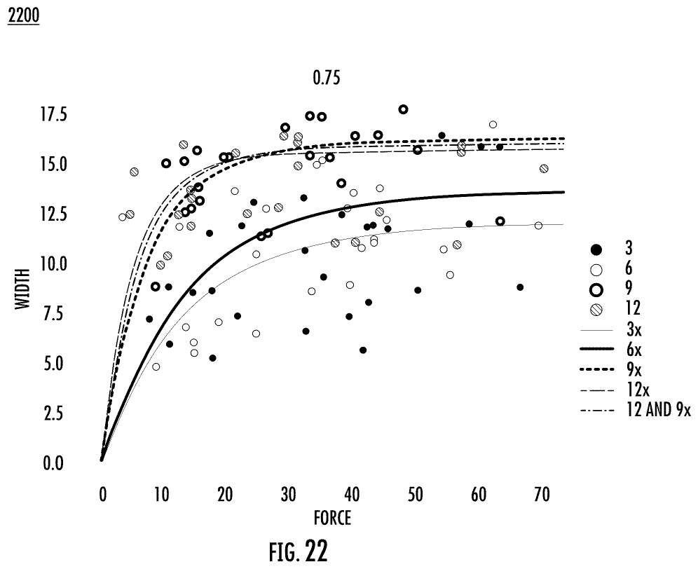

FIG. 22 illustrates a chart 2200 of the width regression. In association with Table 8, below, providing the application numbers and b0 of the regression.

TABLE 8
Width Regression

| Application | |
|---|---|
| of Energy | b0 |
| 3 | 0.06619596059949726 |
| 6 | 0.04068350777427337 |
| 9 | 0.05773424087066371 |
| 12 | 0.07255232013345032 |

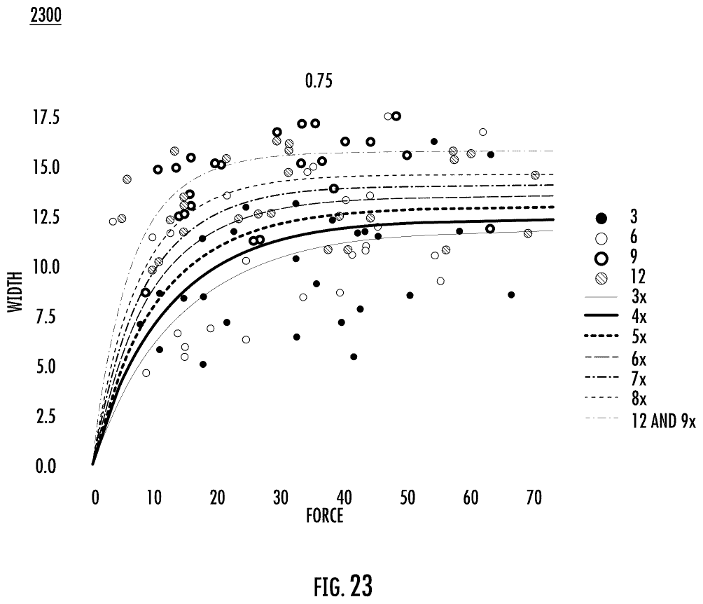

FIG. 23 illustrates a chart 2300 of the width interpolation regression. In association with Table 9, below, providing the application numbers and b0 of the regression.

TABLE 9
Width Interpolation Regression

| Application | |
|---|---|
| of Energy | b0 |
| 3 | 0.06619596059949726 |
| 4 | 0.060790283699534466 |
| 5 | 0.05953802783691534 |
| 6 | 0.059925616582505344 |
| 7 | 0.061113727551866864 |
| 8 | 0.06274345866589048 |

The distance equation is provided as illustrated in Equation 5 below.

$$Log\_result = - ln ( ( distance - x ) / applications \{ distance \geq 6 ; distance \leq 12 \qquad Equation 5$$

where saturation_func=1/(1+exp(−(log_result−ln(max_value)))) and Y=max_value*saturation_func and where max_value=1 with max_value scaling the output to the desired maximum value (1 mm), distance=distance from surface to center of basket, x=recorded depth or width of the lesion, and applications=number of applications.

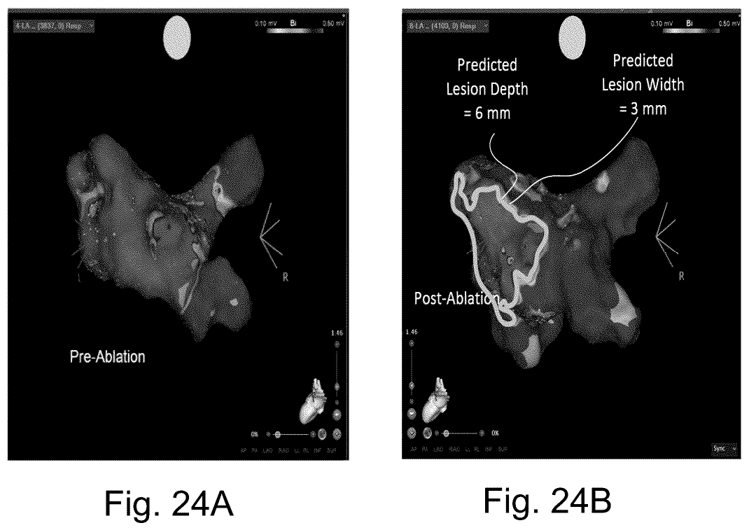

FIG. 24A illustrates an electro-anatomical map prior to ablation with colors indicating functional areas such as for example, red indicative of erratic signals being generated with blue and green denoting zones of ablation.

FIG. 24B illustrates the zones of ablation in red including isolation of the pulmonary veins and spot ablations on the posterior wall and roof of the left atrium where there was previously purple on the pre-ablation map (FIG. 24A). The purple on FIG. 24B represents areas unaffected by ablation. It should be noted that the zones of ablation may indicate either in alphanumeric or pictographic form either or both depth and width of the lesion. The predicted lesion width may be 13 mm for each ablation dot.

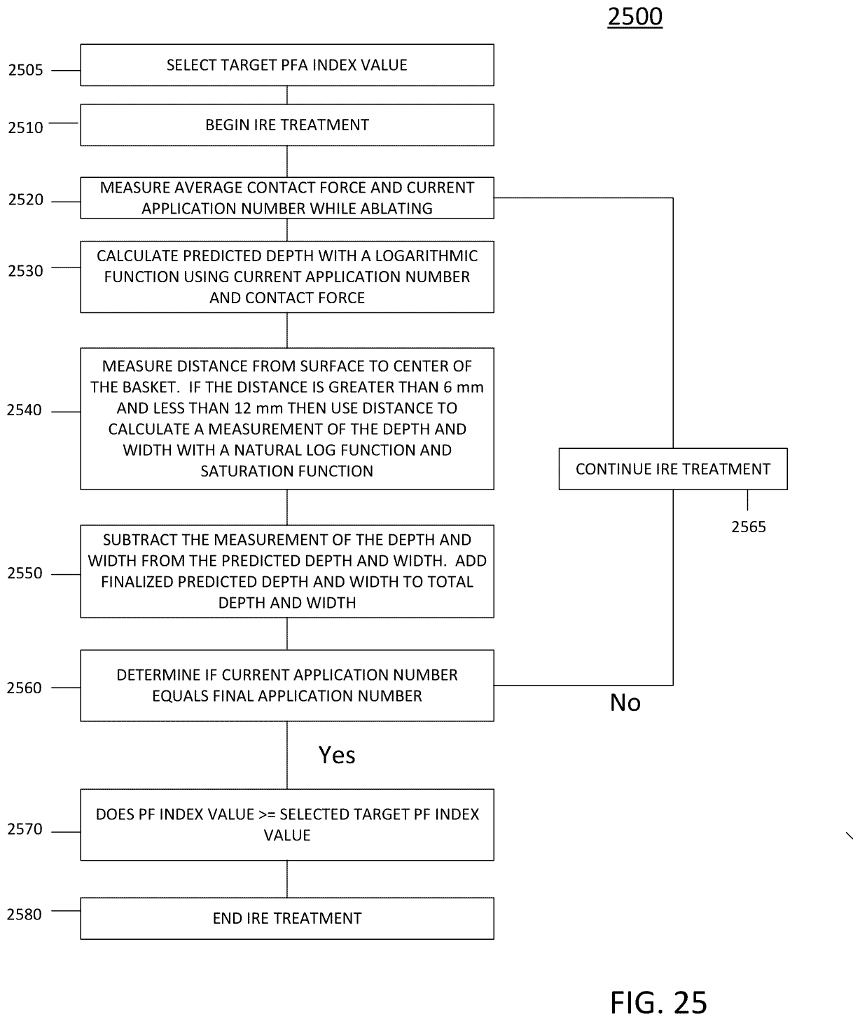

FIG. 25 illustrates a method 2500 according to an aspect of the present invention. At 2505, method 2500 includes selecting a target PFA index value. At 2510, method 2500 includes beginning IRE treatment. At 2520, method 2500 includes measuring the average contact force and current application number while ablating. At 2530, method 2500 includes calculating the predicted depth with a logarithmic function using current application number and contact force.

At 2540, method 2500 includes measuring the distance from surface to center of the basket. If the distance is greater than 6 mm and less than 12 mm then the distance may be used to calculate a measurement of the depth and width with a natural log function and saturation function. This is illustrated by Equation 5.

At 2550, method 2500 includes subtracting the measurement of the depth and width from the predicted depth and width. This may include adding a finalized predicted depth and width to the total depth and width, for example.

At 2560, method 2500 includes determining if the current application is equal to the final application number. If the determining at 2560 is no, then continuing Ire treatment at 2565 occurs and method 2500 reverts to 2520. If the determining at 2560 is yes, then at 2570, method 2500 includes determining if PF index value >=selected target PF index value. At 2580, method 2500 includes ending IRE treatment.

Any of the examples or embodiments described herein may include various other features in addition to or in lieu of those described above. The teachings, expressions, embodiments, examples, etc., described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but, in any order, as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

What is claimed is:

1. A method for determining an ablation lesion zone (width or depth) projected onto an electro-anatomical map, the method comprising:
delivering pulsed energy between at least two electrodes in contact with biological tissues;
storing a magnitude of a contact force as applied by the at least two electrodes to the biological tissues;
calculating one or more ablation zone from the delivering step based on the magnitude of the contact force; and
projecting the one or more ablation zones onto an electro-anatomical map.

2. The method of claim 1 wherein the ablation zone comprises depth or width of an ablated region, the depth or width is calculated from
$$V = Vm - ( Vm - V 0 ) * exp ( - ( b 0 * log ( n ) ) * x )$$
where V is a depth or width of a lesion, x is contact force measured, n is the application number of ablations applied by the electrodes, Vm is the max value of V (of the depth or width) of a probable lesion, V0 is the initial value of the depth or width of the lesion, and b0 is a selectable parameter based on the number of n via a lookup table.

3. The method of claim 1, wherein the determining of the ablation comprises a multi-parameter regression.

4. The method of claim 1, wherein the determining of the ablation comprises a single-parameter regression.

5. The method of claim 1, wherein the electroporation comprising irreversible.

6. The method of claim 1, wherein the electro-anatomical map comprising three-dimensional representation of a heart on a display screen.

7. The method of claim 1, further comprising presenting information about the ablation lesions.

8. The method of claim 1, wherein the ablation application parameter comprises tissue wall thickness, type of tissues nearby and orientation of myocardial fibers.

9. The method of claim 8, wherein the type of nearby tissues comprises at least of myocardium and smooth muscle.

10. The method of claim 1, wherein the projecting provides a viewer of the map with a visual of the ablation lesion including lesion width, lesion depth and cardiac tissue in the zone of electroporation.

11. The method of claim 10, wherein the projecting further includes color coding of the ablation lesion.

12. The method of claim 11, wherein the color-coding denotes at least one of irreversible electroporation and reversible electroporation.

13. The method of claim 10, wherein the size of the visual of the ablation lesion correlates with the size of the lesion.

14. The method of claim 1, wherein the performed regression includes pre-clinical work that relates the ablation parameters to the lesion depth and width.

15. The method of claim 1, further comprising validating the performed regression pre-clinical work that relates the ablation parameters to at least one known lesion depth and width.

16. A system for determining an ablation lesion zone (width or depth) projected onto an electro-anatomical map, the system comprising:
an ablation catheter;
at least one sensor operating in conjunction with the ablation catheter and communicatively connected via hardware including at least one processor; and
a monitor communicatively coupled to the controller operating the ablation catheter and the at least one sensor,
storage to store information representing contact force as applied by at least two electrodes on biological tissues and the number of times energy were delivered;
a controller to calculate one or more zones of electroporation based on a magnitude of contact force and number of times energy were delivered;
the monitor projecting the determined one or more ablation zones onto an electro-anatomical map.

17. The system of claim 16, wherein the processor conducts a multi-parameter regression from predetermined pre-clinical data.

18. The system of claim 16, wherein the processor conducts a single-parameter regression from predetermined pre-clinical data.

19. The system of claim 16, wherein the projecting provides a viewer of the map with a visual of the ablation lesion including lesion width, lesion depth and cardiac tissue in the zone of electroporation.

20. The system of claim 19, wherein the projecting further includes color coding of the ablation lesion.

21. The system of claim 19, wherein the size of the visual of the ablation lesion correlates with the size of the lesion.

22. The system of claim 16, wherein the performed regression includes pre-clinical work that relates the ablation parameters to the lesion depth and width.
